(19) United States
(12) Patent Application Publication
(10) Pub. No.: US 2006/0123495 A1
(43) Pub. Date: Jun. 8, 2006

(54) Knockout mice and their use

(76) Inventors: Donald Gullberg, Uppsala (SE); Evy Lundgren-Akerlund, Bjarred (SE)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 10/510,809

(22) PCT Filed: Apr. 11, 2003
(86) PCT No.: PCT/SE03/00584

§ 371 Date: Oct. 12, 2004

Related U.S. Application Data
(60) Provisional application No. 60/371,731, filed on Apr. 12, 2002.

(30) Foreign Application Priority Data
Apr. 12, 2002 (SE) 0201130-2

(51) Int. Cl. A01K 67/027

(52) U.S. Cl. 800/14

(57) ABSTRACT

Non-human mammals and their progenies comprising an integrin alpha10 gene, integrin alpha11 gene, or both genes are provided, wherein at least a part of the integrin alpha10 gene, the integrin alpha11 gene, or both genes, of said non-human mammal and its progeny has/have been replaced with a DNA sequence comprising at least a portion of one exon of the integrin alpha10 coding sequence, the integrin alpha11 coding sequence, or both coding sequences, linked to a selection marker sequence. Also included are methods for generating said non-human mammals with a disrupted alpha10 gene, a disrupted alpha11 gene or both genes disrupted, as well as the use of said non-human mammals.

### TECHNICAL FIELD

This invention relates to a non-human mammal and its progeny, wherein the expression of alpha10 and/or the alpha11 integrin gene has been suppressed by a targeted disruption of the gene(s). Also contemplated is the use of such non-human mammal(s) and methods for generating said non-human mammal(s).

### BACKGROUND OF THE INVENTION

Integrins

Integrins are heterodimeric transmembrane molecules consisting of large globular extracellular domains formed by alpha- and beta-subunits that bind to specific extracellular matrix (ECM) proteins, and short cytoplasmic domains that interact with cytoskeletal proteins and signalling proteins inside the cell (Hynes (1992) Cell 69(1):11-25). The integrin family comprises to date 24 members, which are the result of different combinations of one of 18 alpha-subunits with one of 8 beta-subunits.

The integrins are composed of non-covalently associated alpha- and beta-chains which connect cells to the extracellular matrix or to other cells (Hynes (1992) Cell. 69(1):11-25). In addition to acting as mechanical links between the cytoskeleton and extracellular ligands, integrins are signal transducing receptors which influence processes such as cell proliferation, cell migration and cell differentiation (Bengtsson et al., (2001) Matrix Biol. 20(8):565-576; Bouvard et al., (2001) Circ. Res. 89(3):211-223; Byzova et al., (1998) Circ. Res. 80(5):726-734).

The Alpha-Chain

One subdivision of integrins can be made based on structural similarities of the alpha-chains. Integrin alpha subunits are known to share an overall identity of 20-40% (Hynes (1992) Curr. Opin. Genet. Dev. 2:621-624). Certain integrin alpha-subunits contain an ˜200 amino acid inserted domain or I-domain in their N-terminal region. In these cases the I-domain is usually identical with the ligand binding site (e.g. alpha subunits 1, 2, 10, 11). In the non-I-domain containing alpha subunits the ligand binding site is less well defined.

The Beta-Chain

Integrins can be grouped into subfamilies based on shared beta-subunits. beta1, beta3, beta4, beta5, beta6 and beta8 integrins are mainly receptors for extracellular matrix molecules, whereas the beta2-and beta7-containing leukocyte integrins take part in cell-cell interactions during immune processes. Most alpha-subunits, with the exception of the alpha-v-subunit, associate with one beta-subunit only. Of the subfamilies with shared beta-chains, the beta1 subfamily has the most members. To date, 12 integrin alpha-chains associated with the beta1-chain have been identified and characterized, alpha1-alpha11 and alphav (Camper-et al (2001) 306(1):107-116).

Together with the alpha-subunit, the N-terminal half of the beta-subunit ectodomain forms part of the extracellular globular integrin head, which is involved in recognition and binding of ECM molecules.

The diversity of the integrins is further increased by isoforms caused by alternative splicing of mRNA of some of the alpha- and beta-subunits and posttranslational modifications of the integrin subunits (Flier and Sonnenberg., (2001) Cell Tissue Res. 305(3):285-298). It is this heterogeneity in the combination of alpha- and beta-subunits that determines the ligand specificity of the integrin.

Binding Entities

Integrins can be subdivided into groups depending on their binding entities. Several integrins bind to extracellular matrix components that contain the RGD sequence, namely fibronectin and vitronectin (Hynes (1992) Cell. 69(1):11-25). Other integrins bind in a non-RGD dependent manner to ligands such as laminin (alpha3-, alpha6- or alpha7-subunits) and collagen (alpha1-, alpha2-, alpha10- and alpha11-subunits). Laminins and collagens also contain RGD sequences, but in the native state these are cryptic and inaccessible.

The collagen-binding integrins can be structurally distinguished from the other extracellular matrix binding integrins by the presence of the I domain in the alpha-subunit known. To date four I domain-containing, collagen-binding integrins have been found in man: alpha1-beta1, alpha2-beta1, alpha10-beta1 and alpha11-beta1. Integrin alpha10-beta1 was described as a type II collagen receptor on chondrocytes (Camper et al. (1998) J. Biol. Chem. 273:20383-20389) and the most recently discovered collagen receptor, integrin alpha11-beta1, was originally described in foetal muscle cells (Velling et al (1999) J. Biol. Chem. 274:25735-25742).

Structural Comparison of Integrins

Structural comparisons of different alpha-subunits have suggested that integrins fold into a so-called 7-bladed beta-propeller structure that forms one globular domain with a ligand binding region on the upper surface (Springer (1997) Proc. Natl. Acad. Sci. USA. 94(1):65-72). Divalent cations i.e. Mg²⁺, Ca²⁺ or Mn²⁺ bind to sites located on the lower surface of blades 4-7 (Humphries (2000) Biochem. Soc. Trans. 28(4):311-339). The extracellular domains of several integrin alpha-subunits also contain an I domain inserted between blades 2 and 3. Structural analysis of the I domains crystallised in the presence of Mg²⁺ has revealed the presence of a characteristic, metal-ion dependent adhesion site motif (MDAS), which is crucial for ligand binding (Plow et al (2000) J. Biol. Chem. 275(29):21785-21788).

In integrin alpha-chains, a less conserved stalk region separates the predicted beta-propeller from the short transmembrane region. This stalk region is possibly involved in transducing conformational changes between the extracellular and intracellular regions, as well as mediating protein-protein interactions. Although integrins take part in cell signalling events, the cytoplasmic tail is short and lacks enzymatic activity. The sequence GFFKR is conserved in a majority of integrin alpha-subunits cytoplasmic tails and has been shown to be important for calreticulin binding (Rojiani et al Biochemistry. (1991) 30(41):9859-66).

Expression of Integrins on Different Cell Types in the Organism

In mammals, integrins are expressed by virtually all nucleated cells and on platelets. Some integrins are expressed widely throughout the body, e.g. the fibronectin receptor alpha5-beta1. Other integrins are more restricted in their expression profile, e.g. the lymphocyte functional antigen 1 (LFA1) alphaL-beta2 is expressed on lymphocytes. Cells express a number of different integrins at the same time on their surface to be able to interact with different ligands in the extracellular matrix or with different receptors on other cells. Some integrins recognise several different ligands, e.g. the alphav-beta3 integrin enables the cell to interact with vitronectin, osteopontin, fibrinogen and other extracellular matrix proteins containing the protein sequence RGD (asparagine-glycine-glutamic acid). On the other hand in some cases there exist also different integrins binding to the same ligand, e.g. the integrins alpha1-beta1, alpha2-beta1, all bind collagen type I. There are, however, differences in signals transduced into the cell generated by binding of these integrins to collagen. Whereas alpha1-beta1 binding to collagen type I leads to down regulation of collagen synthesis in fibroblasts alpha2-beta1 leads to an up regulation of collagen synthesis in fibroblasts (Heino, J., Matrix Biol. (2000) 19(4):319-23).

Chondrocytes

Chondrocytes are the only cell type in cartilage. Articular chondrocytes have been found to express several members of the integrin family, which can serve as receptors for fibronectin (alpha5-beta1), types II and VI collagen (alpha1-beta1, alpha2-beta1, alpha10-beta1), laminin (alpha6-beta1) and vitronectin and osteopontin (alphaV-beta3) (Loesser (2000) Biorheology 37:109-116). alpha2beta1 is a receptor for the cartilage matrix protein chondroadherin (Camper et al. (1997) J. Cell Biol. 138(5):1159-1167).

Diseases in Skeleton and Cartilage

Dysfunction of the chondrocyte and other extracellular matrix-producing cells can lead to the synthesis, turnover or assembly of an abnormal extracellular matrix. This may have severe consequence to the tissue or organ involved resulting in one or more musculoskeletal disorders. Musculoskeletal diseases are common and today generate a high cost to our health society system:
Four out of 10 pensioners experience daily joint pain, but only half have consulted a health professional.
Osteoarthritis is more than 10 times as common as rheumatoid arthritis.
In Sweden, some 15 000 joints are surgically replaced every year because of osteoarthritis, and in the USA more than 500 000.
The prevalence of osteoarthritis increases steeply with age after 50, and this section of the population will double worldwide between 1990 and 2020.

Osteoarthritis and rheumatoid arthritis are the two common joint diseases. While rheumatoid arthritis is considered a systemic multijoint autoimmune disease with a marked inflammation, osteoarthritis is often limited to one or a few joints and with limited signs of inflammation. Both diseases can lead to the destruction of the joint and significant functional limitations for the individual.

Current treatment of rheumatoid arthritis focuses on limiting the pain, inflammation and immune response with drugs that suppress these processes. Recently introduced biological treatments use antibodies or soluble receptors for TNF, one of the key drivers of the disease process. These treatments are often effective, but their effect is limited to the duration of treatment, they are very costly, and they have significant side effects. Even with these shortcomings, the new ‘biologicals’ are regarded as a breakthrough, providing proof-of-concept that the disease process can be modified and the joint protected from further breakdown.

Pharmacological treatments for osteoarthritis as currently practised aim at decreasing pain and improving function by use of analgesics and non-steroidal anti-inflammatory drugs. There is no treatment available that with certainty influences the processes that destroy the joint in osteoarthritis. For end stage osteoarthritis, the best treatment is joint replacement.

Sales of drugs for musculoskeletal disease in 2001 were 15.5 billion US$ in the 13 largest markets. Annual rate of increase in 2001 was 16%, the greatest among all drug groups. Drugs for osteoarthritis and rheumatoid arthritis represent a significant share of this group.

Drug development for rheumatoid arthritis involves additional ‘biologicals’ about to be introduced on the market, and an intense search for small molecule compounds that might provide the same disease-modifying effect as the ‘biologicals’ through the same or other pathways.

Drug development for osteoarthritis follows several paths. On the one hand, better analgesics/non-steroidal anti-inflammatory drugs with less side effects are being sought. On the other hand, it is clear that no treatment exists that can modify the disease process. Intense interest has been focused on protease inhibitors as a means to protect the joint, but no such drug has yet been successful in clinical trials. Attention is now also being brought to the role of inflammation in osteoarthritis, and pathways are explored to identify targets.

In summary, current treatment of osteoarthritis deals only with the symptoms, and when insufficient the joint is replaced. Treatment that will limit the disease process and cartilage destruction is greatly needed, but not yet available. A further long-term goal is to stimulate the regeneration of joint cartilage.

Alpha10

A newly discovered collagen-binding integrin, alpha10beta1, includes the integrin subunit alpha10 (Camper et al., (1998) J. Biol. Chem. 273:20383-20389). The integrin is expressed on chondrocytes and shows a Mᵣ of 160 kDa after reduction when isolated from bovine chondrocytes by collagen type II affinity purification.

Cloning and cDNA sequencing showed that it shares the general structure of other integrin alpha subunits. The predicted amino acid sequence consists of a 1167-amino acid mature protein, including a signal peptide (22 amino acids), a long extracellular domain (1098 amino acids) a transmembrane domain (22 amino acids), and a short cytoplasmic domain (22 amino acids). In contrast to most alpha-integrin subunits, the cytoplasmic domain of alpha10 does not contain the conserved sequence KXGFF(R/K)R. Instead, the predicted amino acid sequence in alpha10 is KLGFFAH. It is suggested that the GFFKR motif in alpha-chains is important for association of integrin subunits and for transport of the integrin to the plasma membrane (De Melker et al. (1997) Biochem. J. 328:529-537). The KXGFF(R/K)R has been shown to interact with the intracellular protein calreticulin (Rojiani et al. Biochemistry (1991) 30(41):9859-66), and interestingly, the calreticulin-null embryonic stem cells are deficient in integrin mediated cell adhesion (Coppolino et al. (1997) Nature 386:843-847).

The extracellular part contains a 7-fold repeated sequence, an I-domain (199 amino acids) and three putative divalent cation-binding sites. The deduced amino acid sequence of alpha10 is 35% identical to the integrin subunit alpha2 and 37% identical to the integrin subunit alpha1. Sequence analysis has revealed that the alpha10 subunit is most closely related to the I domain-containing α subunits with the highest identity to alpha1 (37%) and alpha2 (35%).

A peptide antibody against the cytoplasmic domain of the alpha10 specifically immunolocalises to chondrocytes in tissue sections of human cartilage, showing that the alpha10alpha1 is expressed in cartilage tissue.

Studies in mice have shown that the alpha10 integrin is expressed in various mouse tissues, such as hyaline cartilage of joints, vertebral column, trachea and bronchi (Camper et al. (2001) Cell Tissue Res. 306:107-116). In addition, alpha10 has been found in the ossification groove of Ranvier, in the aortic and atrioventricular valves of the heart and in the fibrous tissue lining skeletal muscle and ligaments. Overall, the distribution was different from that of the collagen-binding integrins alpha1beta1 and alpha2beta1. Expression of alpha10 in mouse appeared on the embryonic day 11.5 at the same time as chondrogenesis began.

Alpha11

The alpha11 integrin has recently been identified on cultured human foetal muscle cells (38). The cloning and characterisation revealed an I-domain containing, beta 1-associated protein.

The open reading frame of the cDNA encodes a precursor of 1188 amino acids. The predicted mature protein of 1166 amino acids contains 7 conserved FGGAP repeats, an I-domain with a MIDAS motif, a short transmembrane region and a unique cytoplasmic domain of 24 amino acids containing the sequence GFFRS.

Alpha11 contains three potential divalent cation binding sites in repeats 5-7. The presence of 22 inserted amino acids in the extracellular stalk portion (amino acids 804-826) distinguishes the alpha11 integrin sequence further from other integrin alpha-chains.

Amino acid sequence comparisons reveal the highest identity (42%) with the alpha10 integrin chain. Immunoprecipitation with antibodies to the alpha11 integrin captured a 145 kDa protein, distinctly larger than the 140 kDa alpha2 integrin chain when analysed by SDS-PAGE under non-reducing conditions.

Fluorescence in situ hybridization maps the integrin alpha11 gene to chromosome 15q22.3-q23, the vicinity of an identified locus for Bardet-Biedl syndrome. Based on Northern blotting, integrin alpha11 mRNA levels are high in adult human uterus and in heart, and intermediate in skeletal muscle and some other tissues tested. During in vitro myogenic differentiation, alpha11 mRNA and protein are up-regulated. Studies of the ligand binding properties show that alpha11beta1 binds collagen type I Sepharose, and in cultured muscle cells alpha11beta1 localizes into focal contacts on collagen type I.

Atherosclerosis

Atherosclerosis and its thrombotic complications are the major cause of morbidity and mortality in industrialised countries. The number of prevalent atherosclerotic cases in 2000 totalled nearly 174 million in the major pharmaceutical markets and this figure will continue to increase with the increase in the ageing population. Still, only a fraction of these show overt signs of the disease. The rest remain undiagnosed until the disease manifests itself symptomatically, in the worst case as a heart attack or stroke. Atherosclerosis is a focal pathological phenomenon characterised by the thickening and hardening of arteries due to the accumulation of lipids (mainly cholesterol esters), carbohydrates, blood products, fibrous tissue and calcium within the vessel wall beginning with the subendothelial space. The gradual build-up of fatty deposits leads to the formation of plaques, which eventually narrow, and block the artery. The causes and detection of such plaque formation has been intensely investigated.

Knockout Animals

Preparation of a knockout animal requires first introducing a nucleic acid construct that will be used to suppress expression of a particular gene into an undifferentiated cell type termed an embryonic stem cell. This cell is then injected into a developing embryo. The embryo is then implanted into a foster mother for the duration of gestation.

Despite the fact that the binding specificities of many of the integrins overlap, the loss of almost any integrin alpha- or beta-subunit leads to biological defects in knockout mice (Bouvard et al. (2001) Circ. Res. 89(3):211-223). These defects vary considerably from embryonic lethality in the disruption of the ubiquitously expressed beta1 integrin gene, leading to the loss of at least 12 different integrin receptors, to the subtle imperfections of the alpha1-knockout mouse.

Inactivation of the alpha7 integrin gene in mouse, as well as mutations in the human ITGA7 gene, has both shown to cause muscular dystrophy affecting mainly muscle attachment points. Of the 11 members of the beta1 subfamily, alpha7 exists as a major integrin alpha-chain associated with the beta1D integrin chain in the adult skeletal muscle sarcolemma. Intriguingly, mutations in the basement membrane protein laminin alpha2-chain cause a more severe disease than those observed for the laminin receptor integrin alpha7beta1. This indicates that other receptors for laminins exist in muscle.

In addition, it is common in tissues in which two integrins are expressed that have similar ligand-binding specificity and properties that some functions maybe compensated by that of the other integrin i.e. the single and double knockouts of the alpha3 and alpha6 integrin subunits (de Arcangelis et al. (1999) Development 126(17):3957-3968).

Although numerous in vitro studies have been performed, the function of integrins on the major cell types of the skeleton (chondrocytes, osteoblasts and osteoclasts) is not well defined and a greater understanding of the role of these integrins is required. In particular, little is known about the biology of the integrins alpha10-beta1 and alpha11-beta1.

In view of the severe disorders that arise due to defects in bone and cartilage and other extracellular matrix related diseases, there is a need in the art to provide in vivo systems and models for studying these disorders, in order to evaluate new and improved treatments for the disorders.

It is thus highly desirable in light of the aforementioned problems and lack of knowledge to develop means and methods for a further understanding of the biology of the integrins and their effect on the skeleton and cartilage, as well as for atherosclerosis, particularly of alpha10-beta1 and alpha11-beta1. In this respect, the present invention addresses this need and interest.

### SUMMARY OF THE INVENTION

In view of the foregoing disadvantages known in the art when studying integrins, particulary alpha10-beta1 and alpha11-beta1 and their effect on the skeleton and cartilage, collagen-containing tissue or tissues containing collagen matrices, musculoskeletal and connective tissue diseases and inflammation, the present invention provides non-human knock-out mammals and its progenies, a method for generating such knock-out non-human mammals, constructs for making such as well as methods and use for modelling different diseases related to the skeleton, joints, muscles, inflammation, cartilage etc.

One object with the present invention is to provide a model system for studying and modulating said integrins alpha10-beta1 and alpha11-beta1 and their effect on the skeleton and cartilage, musculoskeletal and connective tissue diseases and inflammation.

Thus, the present invention provides a non-human mammal and its progeny comprising an integrin alpha10 gene, wherein at least a part of an integrin alpha10 gene of said non-human mammal and its progeny has been replaced with a DNA sequence comprising at least a portion of one exon of the integrin alpha10 coding sequence linked to a selection marker sequence.

Also, the present invention provides a non-human mammal and its progeny comprising an integrin alpha11 gene, wherein at least a part of an integrin alpha11 gene of said non-human mammal and its progeny has been replaced with a DNA sequence comprising at least a portion of one exon of the integrin alpha11 coding sequence linked to a selection marker sequence.

Furthermore, present invention also provides a non-human mammal and its progeny comprising an integrin alpha10 gene and an integrin alpha11 gene, wherein at least a part of an integrin alpha10 gene of said non-human mammal and its progeny has been replaced with 1) a DNA sequence comprising at least a portion of one exon of the integrin alpha10 coding sequence linked to a first marker sequence and 2) a DNA sequence comprising at least a portion of one exon of the integrin alpha11 coding sequence linked to a second selection marker sequence. Still furthermore, the invention provides a method for preparing a non-human mammal and its progeny with a disrupted integrin alpha10 gene, comprising the step of replacing a portion of the integrin alpha10 gene in an embryonic stem cell by homologous recombination with a DNA sequence comprising at least a portion of one exon of the integrin alpha10 coding sequence linked to a marker sequence. The method according to the invention further comprising the steps of providing a knockout construct for integrin alpha10, providing an ES cell line, transformating the ES cell line in b) with the construct in a), selecting transformed ES cell line using a marker sequence, providing a blastocyst, introducing the ES cell line into the blastocyst, transferring the blastocyst to a fostermother non-human mammal, and, allowing an embryo to develop to a chimaeric animal to enable germline transmission of the disrupted integrin alpha10 gene.

Similarly, the invention provides a method for generating the alpha11 knock-out as well as a double knock-out, wherein both alpha10 and alpha11 genes expression are disrupted.

Uses of the different non-human mammals and their progenies of the invention, as well as methods for screening agents for effects in musculoskeletal and connective tissue diseases, atherosclerosis, fibrosis, differentiation of stem cells, such as mesenchymal stem cells, fracture healing, inflammatory diseases, gene therapy are also provided.

### SHORT DESCRIPTION OF DRAWINGS

FIG. 1A shows the targeted disruption of the alpha10 gene with restriction enzyme maps showing the important sites in the generation of a targeted alpha10 allele. X, Xba I; N, Not I. In the targeted alpha10 allele, the gene for the enhanced green fluorescent protein (EGFP) is inserted into exon 1 and replaces the endogenous translational start sequence. The Neo gene is flanked by flox-sites to enable a removal of the Neo cassette. The arrows show the locations for the primer pairs used in PCR for genotyping the mice. The lower part of the figure shows the location of the external probe used for the identification of homologously recombinated ES clones and for genotyping of mice by Southern blot analysis,

FIG. 1B shows Southern blot analysis (top) and PCR (bottom) of DNA isolated from wild-type, heterozygous and homozygous alpha10 mice. The wild-type allele and the homozygously targeted alpha10 alleles give rise to a 10 kb and a 5.5 kb fragment in Southern blot, respectively. In PCR the wild-type allele gives raise to a 370 bp band and the mutant a 210 bp band,

FIG. 2A shows RT-PCR of alpha10 knockout mice in a RT-PCR analysis of the heart from wild-type, heterozygous and homozygous knockout alpha10 mice. Primers are located in exon 1 and exon 6, resulting in a band of 550 bp. No mRNA message is detectable from alpha10 knockout mice. As a positive control, primers specific for beta-actin are used,

FIG. 2B shows surface biotinylation and immunoprecipitation of the alpha10 integrin using rib chondrocytes from wild-type and knockout mice. The alpha10 integrin was not detected on the cell surface of chondrocytes from knockout mice,

FIG. 3 shows immunohistochemical staining of the knee joint from wild-type (A) and knockout alpha10 mice (B). Knee joints were stained for the alpha10 subunit by using an immunofluorescent (Cy3) tagged secondary antibody. Alpha10 was detected in the wild-type knee joint (A) but not in the alpha10 knockout knee joint (B),

FIG. 4 shows length measurements of the tibia and femur in alpha10 knockout mice. Measured length of tibia and femur (at ages 8 weeks, 12 weeks and 1 year) is shown by percent of wild-type length. Alpha10 knockout mice show about 5-10% shorter stature compared to wild-type mice,

FIG. 5 shows haematoxilin/cytochrome staining of tibial growth plate from wild-type (A) and alpha10 knockout (B) mice. By week 8 the morphology of the chondrocytes in the upper proliferative zone of the growth plate exhibit marked changes in the wild-type (A) compared to the knockout mice (B). Chondrocytes from the knockout mice are irregular in size and are rounder in shape compared to the wild-type. In addition, the regular columnar stacking of the chondrocytes in the wild-type is lost in favour of a more random, disorganised proliferative zone,

FIG. 6 shows haematoxilin/eosin staining of the knee joint from wild-type (A) and alpha10 knockout (B) mice. Gross macroscopic changes resembling early fibrillation are observed in the articular cartilage of 1-year-old knockout mice (13). In contrast, wild-type mice (A) of the same age exhibit a normal, unfibrillated articular surface,

FIG. 7 shows immunoprecipitation of alpha10 using alpha10 immune serum. Alpha10 transfected 293 cells were surface biotinylated and subsequently lysed. Immunoprecipitation was carried out using anti-alpha10 cytoplasmic domain antiserum (Lane 1), monoclonal anti-beta1 antibody (Lane 2), mouse non-immune serum (Lane 3) and alpha10 mouse immune serum (Lane 4). The immuno-precipitates were run on a 8% SDS-PAGE and transferred to a PVDF membrane. The blot was stained with avidin-peroxidase conjugate/ECL to detect biotinylated proteins,

FIGS. 8A and 8B shows the targeted disruption of the alpha11 gene. Restriction enzyme maps shows the important sites in the generation of a targeted alpha11 allele,

FIG. 9 shows Southern blot analysis of DNA isolated from wild-type (+/+), heterozygous (+/−) and homozygous (−/−) alpha11mice. The wild-type allele and the homozygously targeted alpha11 alleles give rise to a 8 kb and a 6.5 kb fragment in Southern blot, respectively,

FIG. 9A shows ES cells. Southern blot data showing non-recombinant wild-type ES-clones (+/+) and recombinant heterozygous ES-clones (+/−),

FIG. 9B shows mousetails. Southern blot data of F2-generation showing wild-type (+/+), and mice heterozygous (+/−) and homozygous (−/−) for the targeted allele,

FIG. 10 shows weight curves for integrin alpha11 female and male wild-type and knockout mice. Body weight was measured at different time points for female and male wild-type and knockout mice. For each sex the number of individuals used is indicated together with the number of measured data points,

FIG. 10A shows female Clone 95. Wild-type (7 mice, 19 data points) and knockout (11 mice 24 data points),

FIG. 10B shows male Clone 95. Wild-type (11 mice 30 data points) and knockout (9 mice 28 data points), and

FIG. 11 shows changes in dermal collagen content of the intercapsular skin in wild-type and knockout alpha11 mice. Dermal collagen thickness in intercapsular skin was measured in wild-type (A) and knockout (B) mice.

FIG. 12 shows electron microscopy of the ECM of growth plate from newborn wild-type and alpha10 knockout (mutant) mice.

### DETAILED DESCRIPTION OF THE INVENTION

Definitions

As used herein, the term “knockout” intends to mean a partial or complete suppression of the expression of at least a portion of a protein encoded by an endogenous DNA sequence in a cell.

The term “knockout construct” refers to a nucleic acid sequence that is designed to decrease or suppress expression of a protein encoded by endogenous DNA sequences in a cell.

The term “marker sequence” is herein intended to mean a nucleic acid sequence that is (1) used as part of a nucleic acid construct i.e. the “knockout construct”, to disrupt the expression of the gene(s) of interest, and (2) used as a means to identify those cells that have incorporated the knockout construct into the genome.

The term “selection marker sequence” is herein intended to mean a nucleic acid sequence that is (1) used as part of a nucleic acid construct i.e. the “knockout construct”, and (2) used in selection steps to select for those cells, e.g. embryonic stem cells, that have incorporated the knockout construct into the cell thereby gaining growth advantages over cells not incorporating the knockout construct.

The terms “gene disruption” and “disruption of a gene” refer to insertion of a nucleic acid sequence into one region of the native DNA sequence, e.g. usually one or more exons, and/or the promoter region of a gene so as to decrease or prevent expression of that gene in the cell as compared to the wild-type or naturally occurring sequence of the gene. As with the terms “decrease or prevent expression”, it is intended to mean a partial, or complete block of expression of the gene.

The terms “rodent” and “rodents” refer to all members of the phylogenetic order Rodentia including any and all progeny of all future generations derived there from.

The term “murine” refers to any and all members of the family Muridae, including rats and mice.

The term “progeny” refers to any and all future generations derived and descending from a particular mammal, i.e. a mammal containing a knockout construct inserted into its genomic DNA. Thus, progeny of any successive generation are included herein such that the progeny, the F1, F2, F3, generations and so on indefinitely are included in this definition.

The terms “modulate activity” and “modulating activity” refers to changes in the level of activity of any components of a particular system, e.g. a wild-type or knockout rodent, or in a cartilage, joint or bone disease in said rodent, as compared to the average activity of that component for a particular species. Such a modulation may be an increase or a decrease of the level of activity, as compared to the average activity of that component for a particular species.

The term “musculoskeletal disease(s)” refers to diseases of the muscles and their associated ligaments and other connective tissue and of the bones and cartilage viewed collectively.

The term “gene therapy” refers to treatment of a disease caused by mal-function of a gene, by adding to the cells of the organism a variant or wild-type gene.

Selection of Gene(s) to be Knocked Out

As revealed above, the present invention relates to a non-human mammal and its progeny wherein an integrin gene has been disrupted, i.e. a knockout animal.

According to the invention, the gene to be knocked out will be a gene that is involved, either directly or indirectly, in the modulation of collagen-containing tissue or tissues containing collagen matrices, including cartilage, tendon, ligaments, invertebral discs, cornea, joint or bone formation, fracture healing, and differentiation, musculoskeletal and connective tissue diseases, atherosclerosis, fibrosis, inflammatory diseases, heart valve diseases, activity in differentiation or function of a stem cell, as well as pathological situations, such as different disease stages, in relation to such events.

The gene to be knocked out may be any integrin alpha10 or 11 gene, provided that at least some sequence information on the DNA to be disrupted is available to use in the preparation of both the knockout construct and the screening probes. Preferred genes to be knocked out are the integrin alpha10 or the integrin alpha11 genes in a rodent, such as a mouse.

The Knockout Mice

Gene targeting technology has recently made it possible to generate mice lacking specific integrins and therefore, to gain further insights into the functional role of integrin alpha10beta1 and alpha11beta1, mice generated were disrupted at the alpha10 and alpha11 gene locus.

According to the invention, a non-human mammal and its progeny comprising an integrin alpha10 gene is provided, wherein at least a part of an integrin alpha10 gene of said non-human mammal and its progeny has been replaced with a DNA sequence comprising at least a portion of the integrin alpha10 coding sequence linked to a selection marker sequence.

Further embodiments include wherein the portion of the integrin alpha10 coding sequence is at least a portion of the integrin alpha10 intron, exon, promotor or a mixture thereof.

Even further embodiments include wherein the DNA sequence comprising at least a portion of one exon of the integrin alpha10 coding sequence linked to a selection marker sequence is the DNA sequence included in the vector deposited with the Deutsche Sammlung von Mikroorganismen und Zellkulturen GmbH, Germany, under the accession number DSM 14933.

Further to the invention, a non-human mammal and its progeny comprising an integrin alpha11 gene is included, wherein at least a part of an integrin alpha11 gene of said non-human mammal and its progeny has been replaced with a DNA sequence comprising at least a portion of the integrin alpha11 coding sequence linked to a selection marker sequence.

Further embodiments include wherein the portion of the integrin alpha11 coding sequence is at least a portion of the integrin alpha11 intron, exon, promotor or a mixture thereof.

Even further embodiments include wherein the DNA sequence comprising at least a portion of one exon of the integrin alpha11 coding sequence linked to a selection marker sequence is the DNA sequence included in the vector deposited with the Deutsche Sammlung von Mikroorganismen und Zellkulturen GmbH, Germany, under the accession number DSM 14934.

In addition, since compensatory mechanisms are known, by that of another integrin when a single integrin gene is disrupted, a non-human mammal lacking both the alpha10 and alpha11 subunits has been generated, i.e. a double knockout.

Thus, included within the scope of this invention is a non-human mammal in which two genes, the alpha10 and alpha11, have been knocked out. Such mammals can be generated by repeating the procedures set forth herein for generating each type of knockout mouse, or simply by breeding two mammals to each other, each with the single gene encoding integrin alpha 10 and alpha11 knocked out, and screening for those with the double knockout genotype.

Accordingly, a non-human mammal and its progeny comprising an integrin alpha10 gene and an integrin alpha11 gene is provided, wherein at least a part of an integrin alpha10 gene of said non-human mammal and its progeny has been replaced with 1) a DNA sequence comprising at least a portion of the integrin alpha10 coding sequence linked to a first selection marker sequence and 2) a DNA sequence comprising at least a portion of the integrin alpha11 coding sequence linked to a second selection marker sequence.

Further embodiments include wherein the portion of the integrin alpha10 and alpha11 coding sequence is at least a portion of the integrin alpha10 and alpha11 intron, exon, promotor or a mixture thereof.

The disruption of the gene of interest, i.e. the integrin alpha10 and/or alpha11 is done via insertion of a nucleic acid sequence into one region of the native genomic DNA sequence. Usually one or more exons, and/or the promoter region of a gene is disrupted, which will lead to a suppressed, decreased or prevented expression of that gene(s) in the cell as compared to the wild-type, or naturally occurring, sequence of the gene in the non-human mammal of interest.

The invention thus further provides a knockout non-human mammal and its progeny, wherein expression of the gene(s) of interest is suppressed, due to the disruption of a gene, as compared to a wild-type non-human mammal. The confirmation of the decrease, suppression or prevention of the expression of said gene may be performed according to any of the methods set forth below, i.e. Northern, Southern or Western blot as well as PCR technologies or an otherwise known method to the skilled man in the art, such as e.g. immunoprecipitation.

According to the invention, the complete prevention of the alpha10 and/or alpha11 gene expression is preferred, i.e. a 100% suppression or prevention of the gene expression.

Further embodiments include a knockout non-human mammal and its progeny, wherein expression of the gene encoding integrin alpha10 is decreased, suppressed or prevented as compared to a wild-type non-human mammal. Such a non-human mammal and its progeny may be wherein the alpha10 is suppressed through insertion of a knockout construct comprising at least a portion of one exon of the integrin alpha11 coding sequence linked to a selection marker sequence.

Similarly, a knockout non-human mammal and its progeny is contemplated, wherein expression of the gene encoding integrin alpha11 is decreased, suppressed or prevented as compared to a wild-type non-human mammal. In a specific embodiment, said alpha11 is suppressed through insertion of a knockout construct comprising at least a portion of one exon of the integrin alpha11 coding sequence linked to a selection marker sequence.

In a double knockout, expression from two different genes are decreased, suppressed or prevented due to disruption of said two genes. According to the invention, a non-human mammal and its progeny is provided, wherein expression of the gene encoding integrin alpha10 and the gene encoding integrin alpha11 is decreased, suppressed or prevented as compared to a wild-type non-human mammal. In a specific embodiment of said double knockout, i.e. the non-human mammal and its progeny, is disclosed wherein the alpha10 is suppressed through insertion of a knockout construct comprising at least a portion of one exon of the integrin alpha10 coding sequence linked to a selection marker sequence, and wherein the alpha11 is suppressed through insertion of a knockout construct comprising at least a portion of one exon of the integrin alpha11 coding sequence linked to a second selection marker sequence.

Further embodiments include wherein the alpha10 and/or alpha11 is suppressed through insertion of a knockout construct comprising at least a portion of the integrin alpha10 coding sequence linked to a selection marker sequence.

Still further embodiments include, wherein the portion of the integrin alpha10 and/or alpha11 coding sequence is at least a portion of the integrin alpha10 and/or alpha11 intron, exon, promotor or a mixture thereof.

Still even further embodiments include wherein the knockout construct comprises at least a portion of one exon of the integrin alpha10 or alpha11 coding sequence linked to a selection marker sequence, comprised in a vector deposited with the Deutsche Sammlung von Mikroorganismen und Zellkulturen GmbH, Germany, under the accession number DSM 14933 and DSM 14934 respectively.

In different embodiments of said double knockout, said selection marker sequence may be the same marker sequences as used when the genes are disrupted as single gene knockouts, as described in the embodiments above, or be different. In one embodiment of said non-human mammal and its progeny disclosed according to the invention, the alpha10 or alpha11 genes are disrupted using constructs wherein at least one of the selection marker sequences is the neomycin resistance gene. Other selection marker sequences to use are obvious to the skilled man in the art, and may by such as Zeocine™, gentamycin, hygromycin and puromycin.

In still a further embodiment, a marker sequence is used to identify those cells that have disrupted the gene of interest, i.e. alpha10 and/or alpha11. Examples of suitable marker sequences are sequences coding for green fluorescence protein (GFP) or lac-z. Other markers well known to the skilled man in the art may as well be used.

In one embodiment of the invention, the marker sequence used for alpha10 is GFP (green fluorescence protein).

Still another embodiment for alpha10 includes wherein the marker sequence used for alpha10 is lac-z.

In another embodiment of the invention, the marker sequence used for alpha11 is lac-z.

Still another embodiment for alpha11 includes wherein the marker sequence used for alpha11 is GFP.

The invention also includes production of alpha10 and alpha11 knockout non-human mammals from different species. Preferred embodiments include any species of rodent, including without limiting, rabbits, rats, hamsters, and mice. Preferred rodents are members of the Muridae family, including rats and mice, wherein the mouse is the most preferred embodiment.

Thus, a specific embodiment of the non-human mammal is wherein the non-human mammal and its progeny is a rodent.

Still further embodiments are wherein said rodent is a mouse.

A Method for Pre-Preparing a Non-Human Mammal and its Progeny with a Disrupted Integrin Alpha10 Gene and/or a Disrupted Integrin Alpha11 Gene

The invention also provides a method for preparing a non-human mammal and its progeny with a disrupted integrin alpha10 gene, comprising the step of replacing a portion of the integrin alpha10 gene in an embryonic stem cell by homologous recombination with a DNA sequence comprising at least a portion of one exon of the integrin alpha10 coding sequence linked to a marker molecule.

Such a method may in further embodiments comprise the steps of
a) providing a knockout construct for integrin alpha10,
b) providing an ES cell line,
c) transforming the ES cell line in b) with the construct in a),
d) selecting transformed ES cell line using a marker sequence,
e) providing a blastocyst,
f) introducing the ES cell line into the blastocyst,
g) transferring the blastocyst to a foster mother non-human mammal, and,
h) allowing an embryo to develop to a chimaeric animal to enable germ line transmission of the disrupted integrin alpha10 gene.

Similarly, a method for preparing a non-human mammal and its progeny with a disrupted integrin alpha11 gene is disclosed. Such a method comprises the step of replacing a portion of the integrin alpha11 gene in an embryonic stem cell by homologous recombination with a DNA sequence comprising at least a portion of one exon of the integrin alpha11 coding sequence linked to a marker molecule.

Further the method preparing a non-human mammal and its progeny with a disrupted integrin alpha11 gene, the method may comprise the steps of
i) providing a knockout construct for integrin alpha11,
j) providing an ES cell line,
k) transforming the ES cell line in j) with the construct in i),
l) selecting transformed ES cell line using a marker sequence,
m) providing a blastocyst,
n) introducing the ES cell line into the blastocyst,
o) transferring the blastocyst to a foster mother non-human mammal, and,
p) allowing an embryo to develop to a chimaeric animal to enable germ line transmission of the disrupted integrin alpha11 gene.
   Even further, a method for preparing a non-human mammal and its progeny with a disrupted integrin alpha10 gene and a disrupted integrin alpha11 gene, i.e. a double knockout, is included according to the invention, comprising the steps of

q) replacing a portion of the integrin alpha10 gene in an embryonic stem cell by homologous recombination with a DNA sequence comprising at least a portion of one exon of the integrin alpha10 coding sequence linked to a marker molecule, and,

r) replacing a portion of the integrin alpha11 gene in an embryonic stem cell by homologous recombination with a DNA sequence comprising at least a portion of one exon of the integrin alpha11 coding sequence linked to a marker molecule.

The method for preparing a double knockout may further comprise the steps of
s) providing a knockout construct for integrin alpha10, and providing a knockout construct for integrin alpha11,
t) providing an ES cell line,
u) transforming the ES cell line in t) with the constructs in s) either at the same time or one at a time,
v) selecting transformed ES cell line using at least one marker sequence,
w) providing a blastocyst,
x) introducing the transformed and selected ES cell line into the blastocyst,
y) transferring the blastocyst to a foster mother non-human mammal, and,
z) allowing an embryo to develop to a chimaeric animal to enable germ line transmission of the disrupted integrin alpha10 gene and integrin alpha11 gene.

The above outlined methods are well known to the skilled man in the art of disrupting genes in mammals. Still, these are time-consuming methods, which may need to be repeated several times before success in disrupting a gene and having a germ line transmission is found. Particularly when generating the double knockout, the steps u) to z) may be repeated several times.

The methods are further described in detail in the paragraphs below, as well as in the following examples.

The Knockout Construct and Preparation Thereof

The invention provides a nucleic acid sequence comprising the integrin alpha10 knockout construct. The sequence of the integrin alpha10 knockout construct is retrievable from vector comprising the DNA deposited under the Budapest convention with accession number DSM 14933 by regular DNA sequencing known to the skilled artisan.

Also provided is a nucleic acid sequence comprising the integrin alpha11 knockout construct. The sequence of the integrin alpha11 knockout construct is retrievable from the vector comprising the DNA deposited under the Budapest convention with accession number DSM 14934 by regular DNA sequencing known to the skilled artisan.

The nucleic acid sequence used as the knockout construct to disrupt the gene of interest is typically comprised of 1) nucleic acid, such as deoxyribonucleic acid (DNA) or ribonucleic acid (RNA), from some portion of the gene, e.g. exon sequence, intron sequence, and/or promoter sequence, to be suppressed and 2) a selection marker sequence used to select the presence of the knockout construct in the cell and optionally a marker sequence used to detect the presence of the knockout construct in the cell. The knockout construct is inserted into a cell, and integrates with the genomic DNA of the cell in such a position so as to prevent or interrupt transcription of the native DNA sequence.

Usually, the nucleic acid to be used in the knockout construct will be one or more exon and/or intron regions, and/or a promoter region, but may also be a cDNA sequence provided the cDNA is sufficiently large to provide sufficient complementary sequence for hybridization when the knockout construct is introduced into the genomic DNA of the ES cell.

The nucleic acid sequence to be used to knock out a selected gene can be obtained using methods well known in the art such as those described by Current Protocols in Molecular Biology (Wiley Interscience and Greene (publishers) Ausubel, F. M., Brent R., Kingston R. E., Moore, D. D., Seidman, J. G., Smith J. A., Struhl, K.) or similar books such as (Sambrook et al. (1989) Molecular cloning: A laboratory manual, 2^{nd} edn. Cold Spring Harbor Laboratory Press, New York). Such methods include, for example, screening a genomic library with a cDNA probe encoding at least a portion of the same gene in order to obtain at least a portion of the genomic sequence. Alternatively, if a cDNA sequence is to be used in a knockout construct, the cDNA may be obtained by screening a cDNA library, where the library is cloned into an expression vector, with oligonucleotide probes or antibodies. If a promoter sequence is to be used in the knockout construct, synthetic DNA or RNA probes can be designed for screening a genomic library containing the promoter sequence. Another method for screening known by the skilled man in the art is by database retrieval of sequences from genomic clones e.g. www.ensembl.org/Mus_musculus/, and www.ncbi.nlm.nih.gov/BLAST/. From such databases, also the physical sequence may be achieved as a nucleic acid sample.

Another method for obtaining the nucleic acid to be used in the knockout construct is to manufacture the DNA sequence synthetically, using e.g. a. DNA synthesizer.

The nucleic acid sequence encoding the knockout construct must be generated in sufficient quantity for genetic manipulation and insertion into ES cells. Amplification may be conducted by 1) placing the sequence into a suitable vector and transforming bacterial or other cells that can rapidly amplify the vector, 2) by PCR amplification, or 3) by synthesis with a DNA synthesizer.

A marker sequence may be any sequence that serves the purposes of being an assayable or detectable marker, although typically it will be a nucleic acid sequence encoding a protein that confers a detectable trait on the cell, such as an antibiotic resistance gene or an assayable enzyme not typically found in the cell, whose expression or presence in the genome can easily be detected.

The marker sequence may be operably linked to its own promoter or to another strong promoter from any source that will be active or can easily be activated in the cell into which it is inserted. Where the marker sequence encodes a protein, the marker sequence may also contain a promoter that regulates its expression.

The marker sequence may not need to have its own promoter attached as it may be transcribed using the promoter of the gene to be suppressed. In addition, the marker gene may have a poly A sequence attached to the 3′ end of the gene; this sequence serves to terminate transcription of the gene.

According to the invention, the knockout construct comprises a marker sequence. In one embodiment of the invention, the alpha10 gene has a GFP marker sequence linked to the alpha10 promoter.

In another embodiment of the invention, the alpha11 gene has a GFP marker sequence linked to the alpha11 promoter.

Further embodiments include wherein alpha10 and/or alpha11 has the lac-z linked to the alpha10 promoter or alpha11 promoter.

The DNA sequence to be used in producing the knockout construct according to the invention may be digested with a particular restriction enzyme, or set of enzymes, selected to cut at a location(s) such that a new DNA sequence encoding a marker gene can be inserted in the proper position within this DNA sequence. The proper position for marker gene insertion is that which will serve to prevent expression of the native gene. Said position will depend on various factors such as the location of restriction sites in the sequence to be cut. Other factors to consider are whether an exon sequence or a promoter sequence, or both, is (are) to be interrupted, i.e. the precise location of insertion necessary to inhibit promoter function or to inhibit synthesis of the native exon. Preferably, the enzyme selected for cutting the DNA will generate a longer arm and a shorter arm, where the shorter arm is at least about 300 base pairs (bp).

In one embodiment of the invention, the alpha10 short arm is 1.3 kb and the long arm 10 kb.

In still a further embodiment, the alpha11 short arm is 2.5 kb and long arm 5.5 kb.

In some embodiments, it may be desirable to actually, in the construct or the nucleic acid to be inserted, remove a portion or even all of one or more exons of the alpha10 and/or alpha11 gene to be suppressed. This is to keep the length of the knockout construct comparable to the original genomic sequence when the marker gene is inserted in the knockout construct. In these cases, the nucleic acid in the construct is cut with appropriate restriction endonuclease(s) such that a fragment of the proper size can be removed.

After the nucleic acid sequence encoding the alpha10 and/or alpha11 gene to be suppressed, and that is to be put into the construct, has been digested with the appropriate restriction enzymes, the marker sequence is ligated into said nucleic acid sequence using methods well known to the skilled artisan and described in (Sambrook et al. (1989) Molecular cloning: A laboratory manual, 2nd edn. Cold Spring Harbor Laboratory Press, New York) or introduced to the rest of the knockout construct by PCR technology. If ligation is used, the ends of the DNA fragments to be ligated must also be compatible. This may be achieved by either cutting all fragments with enzymes that generate compatible ends, or by blunting the ends prior to ligation. Blunting is done using methods well known in the art, such as for example by the use of the Klenow fragment of DNA polymerase I to fill in sticky ends. Other ways of introducing compatible ends is by PCR technology, well known to the skilled man in the art.

The ligated, or otherwise linked together, knockout construct may be inserted directly into embryonic stem cells as described below, or it may first be placed into a suitable vector for amplification prior to insertion. Preferred vectors are those that are rapidly amplified in bacterial cells such as the pBluescript II SK vector (Stratagene, San Diego, Calif.) or pGEM7 (Promega Corp., Madison, Wis.).

In one embodiment of the invention, the pBluescript II SK (+) vector is used.

Targeting Strategy for Disrupting Integrin Genes

Targeting by insertion usually occurs by homologous recombination i.e. regions of the knockout construct that are homologous to endogenous DNA sequences hybridize to each other when the knockout construct is inserted into the cell and recombines so that the knockout construct is incorporated into the corresponding position of the endogenous DNA.

The knockout construct nucleic acid sequence may comprise 1) a full or partial sequence of one or more exons and/or introns of the gene to be suppressed, 2) a full or partial promoter sequence of the gene to be suppressed, or 3) combinations thereof. Typically, the knockout construct is inserted into an embryonic stem cell (ES cell) and is integrated into the ES cell genomic DNA, usually by the process of homologous recombination. This ES cell is then injected into, and integrates with, the developing embryo which is further described below.

Transfection of Embryonic Stem Cells

Generally, the embryonic stem cells (ES cells) used to produce the knockout mammal will be of the same species as the knockout mammal to be generated. Thus for example, mouse embryonic stem cells will usually be used for generation of knockout mice.

According to the invention, an ES cell line comprising the integrin alpha10 knockout construct is provided.

Even further, an ES cell line comprising the integrin alpha11 knockout construct is provided.

Still even further, an ES cell line comprising the integrin alpha10 knockout construct and the integrin alpha11 knockout construct, is provided.

Examples of knock-out constructs for the integrin alpha10 and alpha 11 are all retrievable from the two deposited vectors DSM 14933 and DSM 14934 comprising alpha10 and alpha 11 knock-out construct.

Embryonic stem cells are typically selected for their ability to integrate into and become part of the germ line of a developing embryo so as to create germ line transmission of the knockout construct. Thus, any ES cell line that is believed to have this capability is suitable for use herein. Thus, examples of suitable ES cell lines to be used according to the invention are the murine ES cell lines GS1-1 (previously BWE4) (Incyte Genomics, Inc. 3160 Porter Drive., Palo Alto, Calif. 94304 USA) and R1 (Samuel Lunenfeld Research Institute, Room 881, Mount Sinai Hospital, 600 University Avenue, Toronto, Ontario, Canada M5G 1X5). Other murine ES cell lines known to the skilled man in the art may also be used.

The cells are cultured and prepared for DNA insertion using methods well known to the skilled man in the art such as those set forth by Robertson (in: Teratocarcinomas and Embryonic Stem Cells: A Practical Approach, E. J. Robertson, ed. IRL Press, Washington, D.C. (1987)), by Bradley et al. (Current Topics in Devel. Biol., 20:357-371 (1986)), by Hogan et al. (Manipulating the Mouse Embryo: A Laboratory Manual, Cold Spring Harbor Laboratory Press, Cold Spring Harbor, N.Y. (1986)) and by Talts, J. F., Brakebusch, C., and Fässler R, (Meth. Mol. Biol. 129:153-187 (1999)).

Insertion of the knockout construct into the ES cells can be accomplished using a variety of methods well known in the art including for example, electroporation, microinjection, and calcium phosphate treatment

In one embodiment of the invention, the method of insertion is electroporation.

Each knockout construct DNA to be inserted into the cell must first be linearized if the knockout construct has been inserted into a vector. Linearization is accomplished by digesting the DNA with a suitable restriction endonuclease selected to cut only within the vector sequence and not within the knockout construct sequence.

For insertion of the DNA sequence into the ES cells, the knockout construct DNA is added to the ES cells under appropriate conditions for the insertion method chosen. Where more than one construct is to be introduced into the ES cell, DNA encoding each construct can be introduced simultaneously or one at a time.

If the cells are to be electroporated, the ES cells and knockout construct DNA are exposed to an electric pulse using an electroporation machine and following the manufacturer's guidelines for use. After electroporation, the cells are allowed to recover under suitable incubation conditions. The cells are then screened for the presence of the knockout construct.

Screening for the presence of the knockout construct can be done using a variety of methods. Where the selection marker gene is an antibiotic resistance gene, the cells are cultured in the presence of an otherwise lethal concentration of antibiotic. Those cells that survive have presumably integrated the knockout construct. If the selection marker gene is other than an antibiotic resistance gene, a Southern blot of the ES cell genomic DNA can be probed with a sequence of DNA designed to hybridize only to the marker sequence. Finally, if the marker gene is a gene that encodes an enzyme whose activity can be detected, such as the e.g. beta-galactosidase, the enzyme substrate can be added to the cells under suitable conditions, and the enzymatic activity can be analysed.

The knockout construct may be integrated into several locations in the ES cell genome, and may integrate into a different location in each cell's genome, due to the occurrence of random insertion events. The desired location of the insertion is in a complementary position to the DNA sequence to be knocked out. Typically, less than about 1-5 percent of the ES cells that take up the knockout construct will actually integrate the knockout construct in the desired location.

To properly identify and confirm those cells with proper integration of the knockout construct, the DNA can be extracted from the cells using standard methods such as those described by (Sambrook et al. (1989) Molecular cloning: A laboratory manual, 2nd edn. Cold Spring Harbor Laboratory Press, New York), or Current Protocols in Molecular Biology (Wiley Interscience and Greene (publishers) Ausubel, F. M., Brent R., Kingston R. E., Moore, D. D., Seidman, J. G., Smith J. A., Struhl, K.). The DNA may then be probed on a Southern blot with a probe or probes designed to hybridize in a specific pattern to genomic DNA from, in this case, the ES cells digested with (a) particular restriction enzyme(s). Alternatively, or additionally, the genomic DNA can be amplified by PCR with probes specifically designed to amplify DNA fragments of a particular size and sequence, i.e. only those cells containing the knockout construct in the proper position will generate DNA fragments of the proper size.

Injection/Implantation of Embryos

After suitable ES cells containing the knockout construct in the proper location have been identified, the cells are inserted into an embryo. Insertion may be accomplished in a variety of ways, however a preferred method is by microinjection. For microinjection, about 10-30 cells are collected into a micropipette and injected into embryos that are at the proper stage of development to integrate the ES cell into the developing embryo.

The suitable stage of development for the embryo is very species dependent, however for mice it is about 3.5 days. The embryos are obtained by perfusing the uterus of pregnant females. Suitable methods for accomplishing this are known to the skilled man in the art, and are set forth by Bradley et al., supra, and by Hogan et al. (Manipulating the Mouse Embryo: A Laboratory Manual, Cold Spring Harbor Laboratory Press, Cold Spring Harbor, N.Y. (1986)) as well as in Talts, J. F., Brakebusch, C., and Fässler R., (Meth. Mol. Biol. 129:153-187(1999)).

While any embryo of the right age/stage of development is suitable for use, embryos may be male and may have genes coding for a coat colour that is different from the coat colour encoded by the ES cell genes. In this way, the offspring can be screened easily for the presence of the knockout construct by looking for mosaic coat colour, which indicates that the ES cell was incorporated into the developing embryo. Thus, for example, if the ES cell line carries the genes for white fur, the embryo selected may carry genes for black or brown fur. The offspring will then be mosaic in coat colour if the ES cell was successfully incorporated in the embryo.

After the ES cell has been introduced into the embryo, the embryo is implanted into the uterus of a pseudo-pregnant foster mother. While any foster mother may be used, they are typically selected for their ability to breed and reproduce well, and for their ability to care for their young. Such foster mothers are typically prepared by mating with vasectomized males of the same species. The stage of the pseudo-pregnant foster mother is important for successful implantation, and is species dependent. For mice, this stage is about 2-3 days pseudo-pregnant.

Screening for Presence of Knockout Genes in the Non-Human Mammal

If the coat colour selection strategy has been employed, offspring(s) that are born to the foster mother may be screened initially for mosaic coat colour. In addition, or as an alternative, DNA from e.g. tail tissue of the offspring may be screened for the presence of the knockout construct using Southern blots and/or PCR as described above.

Another suitable way of screening for the presence of knockout genes is immunoprecipitation as given in the examples below.

Offspring that appear to be mosaics are then crossed to each other if they are believed to carry the knockout construct in their germ line to generate homozygous knockout animals. If it is unclear whether the offspring will have germ line transmission, they can be crossed with a parental or other strain and the offspring screened for heterozygosity. The heterozygotes are identified by Southern blots and/or PCR amplification of the DNA, as set forth above. The heterozygotes can then be crossed with each other to generate homozygous knockout offspring. Homozygotes may be identified by Southern blotting of equivalent amounts of genomic DNA from mice that are the product of this cross, as well as mice that are known heterozygotes and wild-type mice. Probes to screen the Southern blots can be designed as set forth in the paragraph Transfection of embryonic stem cells above.

Other means of identifying and characterizing the knockout offspring are available. For example, Northern blots can be used to probe the mRNA from the mouse for the presence or absence of transcripts encoding either the gene knocked out, the marker gene, or both. In addition, Western blots can be used to assess the level of expression of the gene knocked out in various tissues of these offspring by probing the Western blot with an antibody against the protein encoded by the gene knocked out, or an antibody against the marker gene product, where this gene is expressed.

Finally, in situ analysis, such as fixing tissue or blood cells from the knockout animal, e.g. a mouse, and labelling with antibody and/or FACS (fluorescence activated cell sorting) analysis of various cells from the offspring may be conducted. This method works well when suitable antibodies to look for the presence or absence of the knockout construct gene product, such as an expressed protein, exist.

Uses of Knockout Non-Human Mammals

The non-human mammal(s) of this invention will have a variety of uses depending on which of the gene or genes that have been suppressed of the integrin alpha10 gene, alpha11 gene or both. The different uses of the non-human mammals according to the invention include not only the whole mouse per se, but also parts of the animal for use in vitro, such as a cell, tissue, organ and bone.

Thus, one use of a non-human mammal and its progeny according to the invention is as a model for modulating activity in musculoskeletal diseases.

Examples of musculoskeletal and connective tissue diseases are arthropathies, such as arthritis, e.g. osteoarthritis (OA), and rheumatoid arthritis (RA); dorsopathies, such as invertebral disc disorders, e.g. thoracic, thoracolumbar and lumbosacral disc disorders; soft tissue disorders such as synovitis, tenosynovitis and other disorders of the synovium, tendon and muscle; osteopathies and chondropathies, such as osteoporosis, osteomalacia, osteochondrosis, chondrodysplasia, and osteochondrodysplasia.

A second use a non-human mammal and its progeny according to the invention is as a model for modulating activity in atherosclerosis

A third use of a non-human mammal and its progeny according to the invention is as a model for modulating activity in fibrosis.

Another use of a non-human mammal and its progeny according to the invention is as a model for modulating activity in differentiation of stem cells. In further embodiments, stem cells may be mesenchymal stem cells, haematopoetic stem cells, epithelial stem cells, neural stem cells, etc.

Still another use of a non-human mammal and its progeny according the invention is as a model for modulating activity in bone fracture healing.

Still another use of a non-human mammal and its progeny according to the invention is as a model for modulating activity in inflammatory diseases.

Further embodiments include wherein the inflammatory diseases are e.g. rheumatoid arthritis and meningitis

Still another use of a non-human mammal and its progeny according to the invention is as a gene therapeutic model for modulating activity of alpha10 and/or alpha11. The expression of the alpha10 and alpha11 in the non-human mammal according to the invention is suppressed, prevented or decreased. By re-introduction of nucleic acid, such as deoxynucleic acid (DNA), ribonucleic acid (RNA), peptidenucleic acid (PNA) or mixtures thereof, the activity may be modulated and the absence of the alpha10 and 11 genes partially or fully compensated. Also, such model may be used for studying over expression of the alpha10 and alpha11 gene expression. Further embodiments include wherein the expression of the alpha10 and alpha11 in the non-human mammal according to the invention is suppressed, prevented or decreased, and wherein integrin alpha10 and 11 genes from other species may be introduced into e.g. a mouse. In one embodiment, human integrin alpha10 and/or 11 nucleic acid is/are in introduced into a knockout mouse according to the invention.

Still another use is wherein the non-human mammal and its progeny according to the invention is used as a model for modulating activity in heart valve diseases.

Still another use of a non-human mammal and its progeny according to the invention, is as a model for preventing, inhibiting, alleviating or reversing activity in musculoskeletal diseases.

Examples of musculoskeletal and connective tissue diseases are arthropathies, such as arthritis, e.g. osteoarthritis (OA), and rheumatoid arthritis (RA); dorsopathies, such as invertebral disc disorders, e.g. thoracic, thoracolumbar and lumbosacral disc disorders; soft tissue disorders such as synovitis, tenosynovitis and other disorders of the synovium, tendon and muscle; osteopathies and chondropathies, such as osteoporosis, osteomalacia, osteochondrosis, chondrodysplasia, and osteochondrodysplasia.

Still another use of a non-human mammal and its progeny according to the invention, is as a model for preventing, inhibiting, alleviating or reversing activity in atherosclerosis.

Still another use of a non-human mammal and its progeny according to the invention, is as a model for preventing, inhibiting, alleviating or reversing activity in fibrosis.

Still another use of a non-human mammal and its progeny according to the invention, is as a model for preventing, inhibiting, alleviating or reversing activity in bone fracture healing.

Still another use of a non-human mammal and its progeny according to the invention, is as a model for preventing, inhibiting, alleviating or reversing activity in inflammatory diseases.

Still another use of a non-human mammal and its progeny according to the invention, is as a gene therapy model for preventing, inhibiting, alleviating or reversing activity of alpha10 and/or alpha11 expression. As used herein, the term “reversing activity” is intended to correlate to the status of the animal upon start. As such, it may have no expression, a decreased expression as compared to a wild-type animal, about wild-type expression or even an expression higher than the wild-type animal, i.e. an over expression. Thus, reversing activity may be both an increase or decrease in the actual alpha10 and/or alpha11 expression.

Still another use is wherein a non-human mammal and its progeny according to the invention is used as a model for preventing, inhibiting, alleviating or reversing activity in heart valve diseases.

The mouse/mice according to the invention may also be used to screen an agent for activity in stimulating, preventing, inhibiting, alleviating or reversing activity in different diseases. Such an agent may be a chemical compound, a drug, or a pharmaceutical compound. It may also be nucleic acid, such as DNA, RNA, a protein or fragments thereof; an antibody or fragments thereof; a peptide, such as a polypeptide or oligopeptide; or a mixture thereof. Also, the agent may be an agent or a mixture of agents retrieved as a natural extract from the vegetable kingdom, or the animal kingdom.

Screening for useful drugs or agents would involve administering the candidate drug or agent over a range of doses to the mouse, and assaying at various time points for a modulating effect(s) of the drug on the disorder being evaluated.

A mammal of the present invention could be used to screen a variety of agents, either alone or in combination, to determine e.g. whether partial or total restoration or activation of a certain activity will occur.

Thus, one use of a non-human mammal and its progeny according to the invention is to screen an agent for activity in preventing, inhibiting, alleviating or reversing activity in musculoskeletal diseases.

Another use of a non-human mammal and its progeny according to the invention is to screen an agent for activity in preventing, inhibiting, alleviating or reversing activity in atherosclerosis.

Still another use of a non-human mammal and its progeny according to the invention is to screen an agent for activity in preventing, inhibiting, alleviating or reversing activity in fibrosis.

Still another use of a non-human mammal and its progeny according to the invention is to screen an agent for activity in stimulating, preventing, inhibiting, alleviating or reversing activity in bone fracture healing.

Still another use of a non-human mammal and its progeny according to the invention is to screen an agent for activity in preventing, inhibiting, alleviating or reversing activity in inflammatory diseases.

Still another use of a non-human mammal and its progeny according to the invention is for the generation of antibodies showing reactivity with alpha10-beta1 or alpha11-beta1, or a part thereof.

One embodiment of such a use is wherein the antibodies generated are polyclonal antibodies.

Another embodiment is wherein the antibodies are monoclonal antibodies.

Still even further embodiments are wherein the integrin alpha10-beta1, alpha11-beta1 or parts thereof is human integin alpha10-beta1, alpha11-beta1 or parts thereof.

Methods for Screening Agents/Compounds in Knockout Non-Human Mammals

According to the invention, several methods are contemplated for screening agents for effects in different diseases. Such methods may include, for example, looking for increased or decreased levels of integrins, e.g. alpha1-2, alpha 5, alpha10-11, alphav; synthesis or degradation of extracellular matrix molecules, such as collagens, e.g. collagen type I, II, VI and IX; non-collagenous proteins, such as chondroadherin (CHAD), cartilage oligomeric matrix protein (COMP), cartilage intermediate layer protein (CILP); and proteoglycans, such as aggrecan, versican, leucine-rich repeat proteins (LRRP); effects on collagen-containing tissue or tissues containing collagen matrices, including cartilage, tendon, ligaments, invertebral discs, cornea, joint or bone; bone fracture healing, bone formation and differentiation, musculoskeletal and connective tissue diseases, dwarfism, atherosclerosis, fibrosis, inflammatory diseases, heart valve diseases, activity in differentiation or function of a stem cell, as well as pathological situations, such as different disease stages, in relation to such events.

Examples of musculoskeletal and connective tissue diseases are arthropathies, such as arthritis, e.g. osteoarthritis (OA), and rheumatoid arthritis (RA); dorsopathies, such as invertebral disc disorders, e.g. thoracic, thoracolumbar and lumbosacral disc disorders; Soft tissue disorders such as synovitis, tenosynovitis and other disorders of the synovium, tendon and muscle; Osteopathies and Chondropathies, such as osteoporosis, osteomalacia, osteochondrosis, chondrodysplasia, and osteochondrodysplasia. Additional examples are tumours in bone and cartilage e.g. osteosarcoma, osteochondroma and chondrosarcoma.

Effects may be assessed by histological analysis of tissues in the non-human mammal, such as the joint, heart valves, blood vessels, cartilage, connective tissue, collagen-containing tissue or tissues containing collagen matrices; morphological analysis if the non-human mammal, such as the dimensions of the skeleton, bone measurements and weight curves; increased or decreased immunoglobulin production, increased or decreased levels of chemical messengers such as cytokines (e.g., interleukins, chemokines and the like), and/or increased or decreased levels of expression of particular genes involved in the particular disorder.

Further, different transcription factors may be analysed for the regulation of protein expression, such as e.g. the transcription factor scleraxis for integrin alpha11 expression.

Still even further, cell cycle regulation may be analysed as a mechanism for control of protein gene expression, such as e.g. as a mechanism for controlling integrin alpha11 gene expression.

A Method for Screening Agents for Effects in Musculosceletal Diseases

One method according to the invention is a method for screening agents for effects in musculoskeletal diseases, comprising the steps of
i) providing a knockout mouse according to the invention,
ii) administering a test agent to said knockout mouse,
iii) determining the effect of said test agent on morphology, on histology, on synthesis and degradation molecules, such as proteins, e.g. integrins, glycoproteins, carbohydrates, lipids, glycolipids, and extracellular matrix molecules, collagen-containing tissue or tissues containing collagen matrices; on regulation of transcriptional and translational activity of the cell,
iv) correlating the effect of said test agent in iii) above with musculoskeletal diseases.

In order to study the progression of a joint disease in an alpha10 or alpha11-deficient mice, the mice may be exposed to an exercise regime, the tread-mill running regimen, to test the effects of mechanical loading on the articular cartilage and compare the results with age-matched, wild-type mice.

Wild-type mice and alpha10 or alpha11 KO mice may then be subjected at different ages e.g. 8 weeks, 12 weeks, to daily running on a 1055M-D40 Exer 6M open treadmill (Columbus Instruments, Columbus, Ohio). Mice are exposed to a training regimen e.g. 6 meters/min for 30 min once a day, 5 days a week and after 8 weeks/12 weeks the mice are killed by CO₂ inhalation. As controls, wild-type and integrin alpha10/11 deficient age-matched mice that were not forced to run are used.

Morphology may also be analysed by X-ray or growth and length curve. Bone growth measurements are performed to look at development of the limbs of a wild-type and alpha10/11 KO mice. Analyses may be performed by anaesthetising the mice with Avertin and by performing X-ray analysis e.g. using a Faxitron MS-20 specimen radiography system for 90 s at 30 kV using X-OMAT TL Kodak diagnostic films. Staining methods can also be used e.g. with alcian blue and alizarin red. The weight and length (nose to anus) of wild-type and alpha10/11-deficient mice are measured from birth onwards at appropriate time intervals.

Further, histological analysis, immunohistochemistry, electron microscopy and staining of skeletons may be analysed for effects. Tissues e.g. articular cartilage, from wild-type and alpha10/11 knockout mice may be collected at various ages for analyses. Tissues for paraffin-embedding may be fixed in 4% paraformaldehyde (PFA) in PBS or in 95% ethanol/5% acetic acid overnight at 4° C. Limbs may be decalcified in 10% EDTA-PBS or 10% formic acid prior to dehydration and embedding. Paraffin sections are cut and tissue for cryosectioning snap-frozen and fixed in e.g. acetone prior to staining. For analysis of cartilage, all sections are treated with 2 mg/ml hyaluronidase for 30 min at 37° C. before antibody staining. Immunofluorescent staining and DAB staining, or equal staining and detection systems, are performed according to standard procedures. Sections may be stained with any known staining methods to visualise the morphology of the tissue. Such methods include staining with haematoxylin/eosin and staining with Safronin 0 to visualise extracellular matrix molecules e.g. proteoglycans and staining to determine the degree of calcification of the tissue e.g. with van Kòssa staining. Other cell types may also readily be detected, e.g. osteoblasts and osteoclasts, using Alkaline Phosphatase and Acid Phosphatase respectively.

Proliferating cells may be detected using 5-bromo-2′-deoxyuridine (BrdU) and an anti-BrdU mAb. Apoptosis may be analysed by using the terminal deoxynucleotide transferase (TdT)-mediated dUTP nick end labelling (TUNEL) method using any commercially available in situ cell death detection kit. Electron microscopy may also be performed on e.g. newborn mice, such as wild-type and alpha10/11 knockout, at various ages to look at e.g. the structure of extracellular matrix molecules within the tissues e.g. collagen structure within the articular cartilage.

Further, biomechanical analysis of tendon structure may be analysed for effects. The effect of the loading regimen on tendon structure may also be investigated. Tendon stiffness may be determined from monotonic failure tests. Flexor digitorum longus (FDL) tendons are then loaded by clamping the ends of a tendon between sandpaper-covered plates. These are preconditioned to constant stiffness and energy loss (hysteresis) and then loaded to failure at 1%/s. Patella tendons are loaded by clamping the patella and quadriceps tendon between sandpaper-covered plates. The tibia is embedded in epoxy up to the distal insertion site within a brass tube. Patella tendons may be loaded to failure at 0.1 mm/s using a suitable loading device. All mechanical tests are conducted at room temperature using for example, a servohydraulic materials testing system (Instron, Canton, Mass.). Stiffness is then calculated from a linear regression of the load deformation curve. Cross-sectional areas of the tendons may be determined under transmitted light microscopy, using e.g. public domain image analysis software (Scion Corp., Frederick, Md.), after fixation of the contralateral limbs in 10% neutral buffered formalin and embedding in poly-methylmethacrylate.

To study the expression patterns of molecules involved in differentiation of the developing limbs in situ hybridisation may be performed on a control and alpha10 or 11 knockout, or both, mice. Limbs from newborn mice may then be collected and fixed in 4% PFA pH 9.5 overnight. Tissues are dehydrated, embedded and cut and mRNA may be detected using any suitable method for RNA detection such as e.g. DIG-labelled RNA probes for extracellular matrix molecules e.g. collagen II, collagen X, Indian Hedgehog Protein (Ihh) and Parathyroid-related Protein (PTHRP)

Cell adhesion and cell spreading may also be stydied for effects. Since the integrins alpha10 and alpha11 are major collagen-binding integrins, cell adhesion and spreading studies may be performed on cells e.g. chondrocytes, isolated from the tissues of the wild-type and alpha10/11 KO mice. Typically cell adhesion studies may be performed in e.g. 48 well plates coated with various extracellular matrix ligands of interest e.g. collagen I, collagen II, fibronectin, vitronectin and BSA. Amounts of ligands, typically 10 ug/ml, are incubated a suitable time, e.g. at 4° C. overnight and freshly isolated cells e.g. chondrocytes seeded at a suitable denity, such as 100 000 cells/well, incubated on the substrates for 1 hour at 37° C. After washing, a suitable substrate e.g. p-Nitrophenyl-N-Acetyl-beta-D-Glucosaminide in buffer with 0.1 M Citrate pH5 and 0.5% Triton X-100 is added and incubated for about 160 minutes. A stop solution, e.g. 50 mM Glycine, 5 mM EDTA pH 10.4, is added and the absorbance read at a suitable wavelength. For cell spreading, freshly isolated cells e.g. chondrocytes, are seeded on different substrates e.g. collagen I, collagen II and fibronectin in a suitable amount, such as 10 microg/ml, in the presence of 0.5% FCS. Cells are incubated at 37° C. overnight or for 3 days and after fixation and permeabilization, the cells are stained antibodies to molecules important for cell structure and movement e.g. Phalloidin (F-actin) or Dnase I (G-actin).

Synthesis and degradation of extracellular matrix molecules e.g. aggrecan, collagen II, from cells from wild-type and alpha10/11 KO mice, e.g. chondrocytes may also readily be determined at the protein level by a number established of methods known in the art. Cells may be isolated from e.g. the joints of mice and placed into cell culture. Cells may be cultured in Dulbecco's Modified Eagles medium (DMEM) containing foetal calf serum (FCS), L-glutamine, penicillin/streptomycin and ascorbate. An appropriate radiolabel may then be added to allow extracellular matrix molecule synthesis or degradation to be monitored using a suitable pulse or pulse-chase protocol e.g. for aggrecan synthesis. Cells may be labelled with 20 microCi/ml ³⁵S-sulphate (Na₂SO₄) or for collagen synthesis ³H-Proline may be added.

Alternatively, quantitative release and degradation of extracellular matrix molecules may be monitored by using dyes e.g. Dimethylmethylene Blue (DMB) that selectively binds to the proteoglycans (Farndale R W et al (1982) Conn. Tiss. Res. 9:247-288) or by ELISA assay where the release of novel epitopes generated by degradation of extracellular matrix molecules is detected by antibodies specific for the neo-epitopes (Caterson B et al. (2000) Matrix Biol. 19(4) 333-44).

Similarly, gene expression may be investigated by isolating mRNA from the cells of the wild-type and alpha10/l 1 KO mice. mRNA isolation may be performed using any suitable kit such as e.g. RNEasy Mini Protocol for the isolation of total RNA (Qiagen). The isolated RNA can then be reverse transcribed by any suitable reverse transcriptase, such as Superscript II RNase H—reverse transcriptase (Invitrogen), according the manufacturer's recommendation. Quantitative PCR may then be performed using any suitable system such as the LightCycler system—Faststart DNA Master SYBR Green I (Roche) with gene-specific primers for the extracellular matrix molecules of interest e.g. collagen II, aggrecan.

A Methd for Screening Agents for Effects in Atherosclerosis

Another method is for screening agents for effects in atherosclerosis, comprising the steps of
i) providing a knockout mouse according to the invention,
ii) administering a test agent to said knockout mouse,
iii) determining the effect of said test agent on morphology, on histology, on synthesis and degradation molecules, such as proteins, such as integrins, glycoproteins, carbohydrates, lipids, glycolipids, and extracellular matrix molecules, collagen-containing tissue or tissues containing collagen matrices; on regulation of transcriptional and translational activity of the cell,
iv) correlating the effect of said test agent in iii) above with atherosclerosis.

The process of atherosclerosis can be viewed as a special type of chronic inflammation where monocytes adhere to the vessel wall and accumulate in the intima. In the presence of low-density lipoproteins (LDL) the monocytes are converted to activated macrophages, which take up lipoprotein particles and become foam cells. This is followed by migration and proliferation of vSMCs within the arterial intima, leading to the great intimal expansion seen in atherosclerotic plaques. The proliferation of the vSMCs is concomitant with a phenotypic modulation of the vSMCs from a contractile to a synthetic phenotype. In addition to proliferation, this phenotypic modulation leads to an increase in the production of extracellular matrix (ECM) molecules discussed in detail by Thyberg et al (Arteriosclerosis. 1990;10(6):966-90). The ECM is critical for the maintenance of vascular integrity and imparts tensile strength, viscoelasticity, elastic recoil and compressibility through the distinct properties of its constituents. Interactions between the ECM and vSMCs are mediated via cell surface receptors such as integrins. Future therapy may be directed towards the modulation of these adhesive interactions mediated by the integrins, which in turn may arrest the development of the atherosclerotic plaque, limit plaque activation and attenuate the thrombotic response accompanying activation.

The integrin alpha10 chain has been shown to be present in the atherosclerotic plaque by the inventors—data is not shown here.

Animal models for studying effects of a drug in atherosclerosis may be used. Calcification frequently occurs in atherosclerotic plaques in humans where it may contribute to plaque rupture and myocardial infarction. An apoE-deficient mouse, which exhibits severe spontaneous arterial atherosclerosis, including cartilaginous metaplasia, and calcification, serves as one model for atherosclerosis and is described in detail by Qiao J-H et al. (Arterioscler Thromb. 1994;14:1480-1497). A knockout mouse of the present invention may be crossed with the apoE-deficient mouse to create a double knockout. These mice may then be used as models for modulating activity and as models for preventing, inhibiting, alleviating or reversing activity and as a method for screening agents for effects in atherosclerosis. The great intimal expansion seen in human atherosclerotic plaques may be reproduced in mice by a periadventitial cuff-induced carotid injury described by Dimayuga P et al. (Biomech. and Biophys. Res. Com. (1999) 264:465-468). By applying this injury model on the knockout mice of the present invention additional models may be created to be used for modulating activity and as models for preventing, inhibiting, alleviating or reversing activity and as a method for screening agents for effects in atherosclerosis.

Determining the effect of said test agent on morphology, or on histopathology may be by e.g. studies on snap-frozen tissue sections fixed in 10% acetone or on formalin-fixed paraffin-embedded tissue sections from e.g. heart, aorta and carotid arteries. Histological stainings may be done with oil red O and haematoxylin and counterstained with fast green for the identification of atheromatous lesions (fatty streaks), arterial calcification, and aortic cartilaginous metaplasia described further in detail by Qiao J-H et al. (Arterioscler Thromb. 1994;14:1480-1497). For confirmation of calcium mineral deposits, representative sections may also be stained by e.g the alizarin red S and von Kòssa techniques.

Effect on synthesis or degradation of proteins e.g. antibodies, cytokines, enzymes, integrins, extracellular matrix components such as collagen type I, II, VI and IX; non-collagenous proteins, such as chondroadherin (CHAD), cartilage oligomeric matrix protein (COMP), cartilage intermediate layer protein (CILP); and proteoglycans, such as aggrecan, versican, leucine-rich repeat proteins (LRRP); and effects on resident or infiltrating cells; and effects on cell surface markers may be by using immunohistochemistry techniques on e.g. snap-frozen tissue sections fixed in e.g. 10% acetone or on formalin-fixed paraffin-embedded tissue sections or using e.g. PCR technique for analysing mRNA levels of above mentioned cells or molecules. Effects on tissue and plasma cholesterol levels (Dimayuga P et al. Biomech. and Biophys. Res. Com. (1999) 264:465-468). Effect on regulation of transcriptional and translational activity in the cell. Effects on periadventitial cuff-induced carotid injury.

A Method for Screening Agents for Effects in Differentiation of Stem Cells

Still another method is for screening agents for effects in differentiation of stem cells, comprising the steps of
i) providing a knockout mouse according to the invention,
ii) administering a test agent to said knockout mouse,
iii) determining the effect of said test agent on morphology, on histology, on synthesis and degradation molecules, such as proteins, such as integrins, glycoproteins, carbohydrates, lipids, glycolipids, and extracellular matrix molecules, collagen-containing tissue or tissues containing collagen matrices; on regulation of transcriptional and translational activity of the cell,
iv) correlating the effect of said test agent in iii) above with effect on stem cells.

In further embodiments of the method, the stem cells may be mesenchymal stem cells, epithelial stem cells, haematopoetic stem cells e.t.c.

A Method for Screening Agents for Effects on Fibrosis

Another method is for screening agents for effects in fibrosis, comprising the steps of
i) providing a knockout mouse according to the invention,
ii) administering a test agent to said knockout mouse,
iii) determining the effect of said test agent on morphology, on histology, on synthesis and degradation molecules, such as proteins, such as integrins, glycoproteins, carbohydrates, lipids, glycolipids, and extracellular matrix molecules, collagen-containing tissue or tissues containing collagen matrices; on regulation of transcriptional and translational activity of the cell,
iv) correlating the effect of said test agent in iii) above with fibrosis.

Fibrosis is defined as the formation of fibrous tissue, the main component of which is the extracellular matrix molecule, collagen. Since the integrins alpha10 and alpha 11 are both members of the collagen-binding integrins and other members of the collagen-binding integrins e.g. alpha1, have been associated with pathological changes in diseases such as kidney fibrosis (Sampson N S et al 2001. J Biol Chem 276(36):34182-8), the alpha10 and alpha11 knockout mice according to the invention provide an ideal model to study the role of these integrins in fibrotic disease e.g. lung fibrosis, liver fibrosis, kidney fibrosis. Several methods are known to be able to induce fibrosis in experimental mice. The methods include the use of organic solvents e.g. vinyl chloride, which can induce skin and lung fibrois, bleomycin, which is used to induce lung fibrosis and carbon tetrachloride used to induce liver fibrosis. Three of these methods and how to determine the effects of agents on fibrosis are described here below;

i) Bleomycin-Induced Lung Fibrosis

Bleomycin-induced lung fibrosis is described by Lasky J A et al (1998 Am J Physiol 275:L365-L371). Integrin alpha10 or 11 knockout mice may be rendered insensitive with tribromoethanol intraperitoneally before sterile tracheal cut-down surgery. An amount of Bleomycin, e.g. four units per kilogram, in 0.9% NaCl may be administered into the tracheal lumen. A similar volume of sterile 0.9% NaCl is instilled into age-matched, control (wild-type) mice. After administration of bleomycin, the neck wound is closed with a clip, and the animals are allowed to recover from the anaesthesia.

At designated time points after exposure (e.g. 8, 16, and 30 days), the mice are anaesthetized with an intraperitoneal injection of tribromoethanol. After ligation of the abdominal aorta, the chest cavity is exposed. The lungs may then be removed for further analyses i.e. collagen content analysis e.g. by hydroxyproline measurement as described by Kivirikko, K I et al (1967 Anal. Biochem. 19:249-255 or with a kit such as the Sircol Collagen Assay Kit (Biocolor Ltd, Belfast UK).

Lung tissue may be perfused with formalin for embedding in paraffin for subsequent histological analysis e.g. collagen staining with picrosirius. Lungs may also be used for RNA preparation to look at differential gene expression of matrix and matrix-remodelling proteins e.g. collagens, laminins, integrins and fibronectin and the protease and protease inhibitors e.g. MMP-1, TIMP-1 and PAI-1 as well as cytokines and growth factors e.g. transforming growth factors and insulin-like growth factors.

ii) Carbon Tetrachlroride-Induced Fibrosis

Carbon Tetrachlroride-induced fibrosis is described by Yu C et al (2002 Am J Path 161(6):2003-2010). For the study of acute CCl4-induced liver damage, a single dose e.g. 2.0 ml/kg of body weight (2:5 v/v in mineral oil) may be administered by intraperitoneal (IP) injection into wild-type or alpha10 or alpha11 knockout mice. For the study of chronic CCl4-induced liver damage, a dose of e.g. 2.0 ml/kg of body weight of CCl4 may be administered IP twice a week. Livers are then excised for analysis after the mice have been weighed, anaesthetized, and exsanguinated.

Further analyses may then be performed on the excised livers e.g. to determine the collagen content e.g. by hydroxyproline measurement or with kits, such as the Sircol Collagen Assay Kit. Tissue may be perfused with formalin for embedding in paraffin and histological analysis e.g. by per forming collagen staining on the sections with picrosirius.

Liver may also be used for RNA preparation to look at differential gene expression of matrix and matrix-remodelling proteins e.g. heparan sulphate proteoglycans, collagens, integrins the protease and protease inhibitors e.g. MMP-1, TIMP-1 and PAI-1 as well as cytokines and growth factors e.g. transforming growth factors and fibroblast growth factors.

iii) Kidney Fibrosis

Kidney Fibrosis is described by Sampson N S et al (2001 J Biol Chem 276 (36) 34182). Most types of progressive renal disease are characterized by kidney failure associated with glomerular and interstitial fibrosis. Within the glomerulus, mesangial matrix expands, and there is increased deposition of extracellular matrix components, including collagen. Glomerular filtration and renal blood flow is increasingly impaired, tubules atrophy, and a general inflammatory response to the tissue damage ensues. The progressive loss of glomerular function terminates in tubulointerstitial fibrosis. The aetiology of kidney fibrosis is many-fold, ranging from disease-associated secondary pathology, e.g diabetic nephropathy, to autosomal mutations of basement membrane proteins.

One such hereditary basement membrane disorder is Alport's syndrome, a result of mutations in the collagen4A5, collagen4A3, or collagen4A4 genes described by Lemmink, H. et al ((1994) Hum. Mol. Genet. 3:1269-1273). There are several animal models for Alport's syndrome, one of which is a collagen 4A3 gene knockout in mouse (Cosgrove, D et al. 1996 Genes Dev. 10:2981-2992). This model may be very useful for studying both the tissue pathology of the disease as well as the underlying gene expression changes that may drive the disease.

Alport's syndrome (col4A3−/−) mice have been described previously (Lemmink, H. et al (1994) Hum. Mol. Genet. 3:1269-1273). These mice are on a 129 Sv/J background and may be further crossed with the alpha10 or alpha11 knockout mice of the present invention. The genotype of the mice may be determined by PCR. At the desired age (e.g. 4 or 7 weeks) mice may be sacrificed, and e.g. the kidneys may be dissected and frozen immediately in liquid nitrogen or by the usual methods for preparing tissue sections e.g. kidneys are embedded in OCT (Tissue Tek) and frozen in liquid nitrogen. Cryostat sections, e.g. 5 μm, may be cut and fixed in ice-cold acetone and after blocking in 3% bovine serum albumin, phosphate-buffered saline. Sections may be stained with the antibodies e.g. to matrix and matrix-remodelling proteins, protease and protease inhibitors, growth factors or monocytes/macrophage markers.

Kidneys may also be used for RNA preparation to look at differential gene expression of matrix and matrix-remodelling proteins e.g. collagens, laminins, integrins and fibronectin, the protease and protease inhibitors e.g. MMP-1, TIMP-1 and PAI-1 and growth factors e.g. endothelin-1 and insulin-like growth factors, monocyte/macrophage-derived genes e.g. MCP-1, macrophage-inducible protein (IP-10), macrophage colony-stimulating factor and macrophage mannose receptor. Kidneys may be homogenized in Trizol at 4° C., and the total RNA may be extracted. The RNA may then be further purified with an RNA isolation protocol, such as a Qiagen RNeasy kit, according to the manufacturer's instructions (Valencia, Calif.). The total RNA isolated may be reverse transcribed using any suitable reverse transcriptase system for use in quantitative PCR using e.g. any system for quantitative PCR, e.g. the LightCycler system—Faststart DNA Master SYBR Green I (Roche) with gene-specific primers for the genes mentioned above.

A Method is for Screening Agents for Effects in Inflammatory Diseases

Still another method is for screening agents for effects in inflammatory diseases, comprising the steps of
i) providing a knockout mouse according to the invention,
ii) administering a test agent to said knockout mouse,
iii) determining the effect of said test agent on morphology, on histology, on synthesis and degradation molecules, such as proteins, such as integrins, glycoproteins, carbohydrates, lipids, glycolipids, and extracellular matrix molecules, collagen-containing tissue or tissues containing collagen matrices; on regulation of transcriptional and translational activity of the cell, correlating the effect of said test agent in iii) above with inflammatory diseases.

One example of an inflammatory disease is e.g. Rheumatoid arthritis.

Rheumatoid arthritis is a disease characterized by a tissue-specific inflammatory attack directed to peripheral joints that ultimately causes a destruction of cartilage and bone. Collagen-induced arthritis is an animal model for Rheumatoid arthritis which can be induced in genetically susceptible animals (Holmdahl R et al. (1990) Immunological Reviews 118: 193-232) (Genetic Analysis of Mouse Models for Rheumatoid Arthritis in: Human genome methods, (1998) chapter 11 pp 215-238 CRC Press NY, Ed. Adolph K W). The DBA/1 mouse is an example of a strain that is susceptible for collagen II-induced arthritis and a knockout mouse of the present invention may be back-crossed to DBA/1 background and then used as a model to study Rheumatoid arthritis. Collagen-induced arthritis is induced in the knockout mouse of the present invention on a DBA/1 background by immunization with heterologous or autologous type II collagen (Holmdahl R et al. (1990) Immunological reviews 118: 193-232) or passively with type II collagen immune serum or monoclonal antibodies to collagen II pathogenic epitopes (Holmdahl R et al. (1990) Scand. J Immunol. 31:147-157, Holmdahl et al. (1991) Autoimmunity 10:27-34).

These mice may be used as a model for modulating activity and as a model for preventing, inhibiting, alleviating or reversing activity and as a method for screening agents for effects in inflammation and musculoskeletal and connective tissue disease. Examples of for determining effects of said agent on disease development are visual scoring of activity and severity of the inflammation in paws and ankles and microscopic scoring of the paws and ankles including evaluation of activity and severity of the arthritis (Genetic Analysis of Mouse Models for Rheumatoid Arthritis in: Human genome methods, (1998) chapter 11 pp 215-238 CRC Press NY, Ed. Adolph K W). For evaluation of healing processes Safranin-O staining may be used which includes visualisation of both new cartilage and bone formation. Effect on synthesis or degradation of proteins e.g. antibodies, cytokines, enzymes, integrins, extracellular matrix components such as collagen type I, II, VI and IX; non-collagenous proteins, such as chondroadherin (CHAD), cartilage oligomeric matrix protein (COMP), cartilage intermediate layer protein (CILP); and proteoglycans, such as aggrecan, versican, leucine-rich repeat proteins (LRRP); and effects on resident or infiltrating cells; and effects on cell surface markers using immunohistochemistry techniques on snap-frozen tissue sections fixed in 10% acetone or on formalin-fixed paraffin-embedded tissue sections or using PCR technique for analysing mRNA levels of above mentioned cells or molecules. Effect on circulating proteins e.g. auto-antibodies and cytokines by enzyme-linked immunosorbent assay (ELISA) standard techniques. Effect on antibody producing plasma cells in lymph nodes or spleen may be analysed by ELISPOT technique and effects on T cell reactivity may be analysed with proliferation assay and ELISPOT may be used for analysis of cytokine production capacity. Effects on regulation of transcriptional and translational activity in the cell may aslo be analysed by e.g. PCR and/or in situ hybridisation.

A Method for Screening Agents for Effects on Bone Fracture

Still another method is for screening agents for effects in bone fracture healing, comprising the steps of
i) providing a knockout mouse according to the invention,
ii) administering a test agent to said knockout mouse,
iii) determining the effect of said test agent on morphology, on histology, on synthesis and degradation molecules, such as proteins, such as integrins, glycoproteins, carbohydrates, lipids, glycolipids, and extracellular matrix molecules, collagen-containing tissue or tissues containing collagen matrices; on regulation of transcriptional and translational activity of the cell,
   iv) correlating the effect of said test agent in iii) above with bone fracture healing.

The biology of fracture healing may be studied in mice by a standardized experimental fracture in mouse tibia as described by Hiltunen A et al. (J Orthopaedic Res. (1993) 11:305-312) and Ekholm E et al. (Am J Pathol (2002) 160:1779-1785).

Determining the effect of said test agent on the morphology by radiographic, chemical and mechanical analyses (Hiltunen A et al. J Orthopaedic Res. (1993) 11:305-312). Radiographs may be examined for the fracture pattern and the size of the callus may be measured. For chemical analyses the fracture callus may be dissected out and measured, weighed, cut to pieces, lyophilized and analysed for e.g. nitrogen, hydroxyproline and calcium contents. Mechanical properties of the fractures may be determined with a four point bending technique in a universal testing device (Alwetron; Lorenzen and Wettre, Stockholm, Sweden). Load-deformation curves may be recorded and analysed for ultimate bending stiffness and bending load. Effects on the synthesis of the three major tissue components—undifferentiated mesenchymal tissue, cartilage and new bone—that are synthesised during the fracture healing may be measured using histological techniques (Ekholm E et al. Am J Pathol (2002) 160:1779-1785). Histological evaluation may be performed on decalcified and paraffin embedded fractures stained with van Gieson and haematoxylin and eosin or safranin and the areas of the major tissue components may then be measured, number of chondrocyte nuclei may be counted. Effect on the composition of the newly synthesised tissue may be performed by immunohistochemistry or RNA extraction and Northern analyses (Ekholm E et al. Am J Pathol (2002) 160:1779-1785). Immunohistochemistry analyses may be carried out on formalin-fixed paraffin-embedded sections or on frozen tissue sections fixed in 10% acetone with antibodies to different extracellular matrix components such as collagen type I, II, VI and IX; non-collagenous proteins, such as chondroadherin (CHAD), cartilage oligomeric matrix protein (COMP), cartilage intermediate layer protein (CILP); and proteoglycans, such as aggrecan, versican, leucine-rich repeat proteins (LRRP). Effect on bone marrow-derived mesenchymal stem cells (MSCs) (Ekholm E et al. Am J Pathol (2002) 160:1779-1785). Bone marrow is flushed from the bone and for colony-forming analyses the cells are plated on tissue culture plates. Osteoblast-committed MSCs may be differentiated from pluripotent progenitors by staining citrate/acetone/formalin fixed cells for alkaline phosphatase and counterstain with phenol red.

A Method is for Screening Agents for Effects in Gene Therapy

Still another method is for screening agents for effects in gene therapy of alpha10 and/or alpha11, comprising the steps of
i) providing a knockout mouse according to the invention,
ii) administering a test agent to said knockout mouse,
iii) determining the effect of said test agent on morphology, on histology, on synthesis and degradation molecules, such as proteins, such as integrins, glycoproteins, carbohydrates, lipids, glycolipids, and extracellular matrix molecules, collagen-containing tissue or tissues containing collagen matrices; on regulation of transcriptional and translational activity of the cell,
   v) correlating the effect of said test agent in iii) above with expression of alpha10 and/or alpha11.

In one embodiment, the method include knockout mice according to the invention being further tragenic for human alpha10, alpha11 or both.

One embodiment of said method for screening agents for effects in gene therapy is wherein the test agent is selected form the group consisting of deoxynucleic acid, ribonucleic acid, PNA, and mixtures thereof.

The alpha10 and alpha11 knockout mice, or the double knockout mouse, may be used to study the effect of delivering the alpha10 or alpha11 gene back to the mice of the present invention.

Gene therapy may thus be performed using e.g. a viral, such as retroviruses, adenoviruses, adeno-associated viruses (AAV), herpes simplex virus and lentivirus; or non-viral delivery system, such as the use of naked DNA, cationic liposomes, cationic lipids and polymers as well as DNA/cationic liposome/polycation complexes, for the in vivo delivery of genes such as alpha10 and alpha11, or both, directly to the cells of interest in the target tissue.

Examples of other suitable genes that can be co-delivered along with the alpha10 or alpha 11 genes include growth factors such as insulin-like growth factor-1 (IGF-1), transforming growth factor-beta (TGF-β), fibroblast growth factors, and bone morphogenic proteins, transcription factors such as SOX-9, certain signalling molecules such as SMADs and molecules that inhibit apoptosis such BCL-2, enzyme inhibitors such as metalloproteinase inhibitors, promoters for genes such as collagens e.g. collagen type II.

A Method for Generating Antibody-Hybridomas Reactive to Alpha10Beta1 and Alpha11Beta1

Still another method is for generating antibody-producing hybridomas reactive to integrin alpha10-beta1 or alpha11-beta1 or parts thereof, comprising the steps of
i) immunizing a mouse with integrin alpha10-beta1 or parts thereof, or integrin alpha10-beta1 or parts thereof,
ii) boosting the immunized mouse in i) above with the immunized antigen,
iii) sacrifying the immunized mouse,
iv) preparating single cell suspension cells from the sacrificed mouse in iii),
v) fusing the single cell suspension in iv) above with a tumour cell line to generate hybridoma cells,
vi) screening the hybridoma cells for reactivity to the immunized antigen.
   A further embodiment of the method is further comprising the steps of limiting dilution of screened, fused hybridoma cells in vi) above, thereby generating monoclonal hybridoma cells.

Alternative methods can be used for the production of monoclonal antibodies specific for alpha10 and alpha11 integrins using the alpha10 and 11 knockout mice.

In one method, alpha10 and 11 knockout mice are immunized with recombinant alpha10 or 11 or parts thereof, purified from an alpha10 or 11, or parts thereof, expressing cell lines. Hybridoma cell lines are generated by transfecting HEK 293-EBNA cells with the expression vector pCEP4 coding for His-tagged alpha10 or 11 or parts thereof, expressing cell lines, alone or fused to alkaline phosphatase (AP) or glutathione transferase (GST). Mice are immunized intramuscularly with e.g. an alkaline phosphatase fusion protein of alpha10 or alpha11 mixed with the mouse adjuvant Immuneasy (Qiagen) or with alpha11-glutathione transferase fusion protein mixed with the mouse adjuvant Immuneasy (Qiagen). Fifteen days later the mice are boosted with the same antigen. Mice are boosted further with 2 or 3 boosts administered subcutaneously at the base of tail at 2 week intervals.

Two days after the last immunization, spleen cells from the mice are fused with NSO myeloma cells using polyethylene glycol. Fused cells are seeded in a 96-well microplate and grown in DMEM/F12 (Invitrogen) medium containing BM Condimed HI (Roche) and HAT (hypoxanthine, aminopterin, thymidine mixture Sigma) selection.

Hybridoma cell clone supernatants are tested for anti-alpha10 and alpha11 antibody production by their ability to bind to immobilized alpha10 or alpha 11 protein or parts thereof, respectively, by e.g. ELISA and by binding to a cell line expressing alpha10beta1 or alpha11beta1 in e.g. FACS analysis.

An alternative method for producing monoclonal antibodies to the integrins alpha10beta1 and alpha11beta1 is by immunization of alpha10 or alpha11-integrin-deficient mice with mouse C2C12 cells or wild-type chondrocytes or fibroblasts. For immunization, cells such as mouse C2C12 cells expressing either the human alpha10 chain or the human alpha11 chain are suspended in adjuvant before subcutaneous injection. After three booster injections, spleen cells are fused with SP/2 myeloma cells, and the resulting hybridoma cells are cloned. Supernatants are screened by immunofluorescence on cartilage sections, and clones that stain specifically the chondrocytes of the articular cartilage are subcloned and then grown to mass culture.

A Method for Creating a Transgenic Mouse

The knockout mouse models according to the invention for integrin alpha10 and alpha11 may also be used to further generate transgenic mice containing the human integrin alpha10 chain and the human integrin alpha11 chain, or both. Such trangenic mice mice may be used for the purpose of screening antibodies, peptides, and small molecules for the treatment of diseases as outlined and exemplified herein.

To generate a transgenic mouse containing either the human integrin alpha10 chain or the human integrin alpha11, or both, chain the following procedure described in brief below can be used.

Embryonic stem cells (ES cells) used to generate the alpha10 and alpha11 knockout mice, or the double knockout mice, of the present invention are used for the generation of transgenic mice comprising human alpha10, alpha11 or both genes. Embryonic stem cells (ES cells) are harvested from the inner cell mass (ICM) of mouse blastocysts. Recombinant DNA methods are used to construct molecules of DNA containing i) the desired structural gene i.e. the human integrin alpha10 gene or human integrin alpha11 gene, ii) vector DNA to enable the molecules to be inserted into host DNA molecules, iii) promoter and enhancer sequences to enable the gene to be expressed by host cells. Recombinant DNA methods are well known in the art and further described in e.g. Current Protocols in Molecular Biology (Wiley Interscience and Greene (publishers) Ausubel, F. M., Brent R., Kingston R. E., Moore, D. D., Seidman, J. G., Smith J. A., Struhl, K.) or similar books such as (Sambrook et al. (1989) Molecular cloning: A laboratory manual, 2^{nd} edn. Cold Spring Harbor Laboratory Press, New York).

The targeting vector containing the DNA of interest is normally electroporated into the ES cells. Typically the targeting vector is designed in which a positive selectable marker e.g. the neo gene (which confers resistance to G418 or neomycin) is flanked by larger stretches of cloned genomic DNA homologous to the target gene. To assist in the elimination of ES cells that incorporate the targeting vector by random insertion rather than homologous recombination, the targeting vector may be constructed with a negatively selectable marker e.g. herpes virus thymidine kinase (tk gene), which confers sensitivity to the drug ganciclovir at one end.

Successfully transformed cells may then be selected in the following manner:
Cells that fail to take up the vector are killed if exposed to neomycin.
Cells in which the vector was randomly inserted are resistant to neomycin but are killed by gancyclovir (in the presence of the tk gene gancyclovir is toxic to cells).
The ES cells that survive in the selection medium form distinct colonies, or clones and Southern blotting or PCR is used to identify the successful gene-targeting.

Appropriately targeted cells, generally about 10-15, are then injected by microinjection into blastocysts and subsequently surgically transferred into the uterus of a pseudopregnant mouse.

Tissue may be removed from e.g. the tail of a mouse to analyse the DNA for presence of human integrin alpha10 or alpha11 gene.

After subsequent matings with wild-type and chimeric mice, F1 heterozygous mice are generated and bred to produce, heterozygous, homozygous and wild-type mice.

### EXAMPLES

### Example 1

### Disrupting the Integrin Alpha10 Gene

Integrin Alpha 10 Knockout Mouse—Targeting Strategy and Generation of the Knockout Construct

A human cDNA probe, corresponding to nt-32-nt 801 (GenBank™/EBI Data Bank accession number AF074015) if integrin alpha10, was used to isolate several overlapping clones (Bengtsson et al (2001) Matrix Biology 20(8):565-576) from a 129SvJ mouse genomic cosmid library (Evans and Kaufman (1981) Nature 292:154-156). A 6 kb Hind III fragment, containing exon 1 (with the ATG start codon) to exon 7, was subcloned into Bluescript SK vector, generating p13. From p13, a 1.3 kb Hind III-Nco I fragment was ligated with a Nco I-Xba I fragment of the Neo gene, before adding a Not I-linker in the 3′ Xho I site of the plasmid. This construct 1 was then moved with Not I and Xba I and inserting it into the backbone of the plasmid pBS EGFP loxP Neo loxP into these sites, leading to construct 2.

A 2.1 kb Nco I fragment, containing the complete EGFP gene (enhanced green fluorescent protein gene) and the rest of the Neo gene from plasmid pBS EGFP loxP Neo loxP was inserted into the Nco I site of construct 2 and orientation determined. The Neo gene in this construct is flanked by loxP sites, making a future deletion of the Neo gene possible.

The ATG start codon of alpha10 is located immediately 3′ of the Nco I site and so is the ATG codon for the EGFP gene. By inserting the EGFP into the Nco I site, the endogenous ATG codon for alpha10 is replaced by the ATG for EGFP. By inserting a Xho I-linker in the unique Not I site 3′ of construct 3, the entire construct could be moved by Xho I digest and ligated into the Xho I site of plasmid p1 1 (Bengtsson et al (2001) Matrix Biology 20(8):565-576), generating the targeting vector. The plasmid p11 contains exon 2 to 25 of the alpha10 gene and has a unique Not I site 3′ in the pBS, which was used to linearize the targeting vector before electroporation of ES cells.

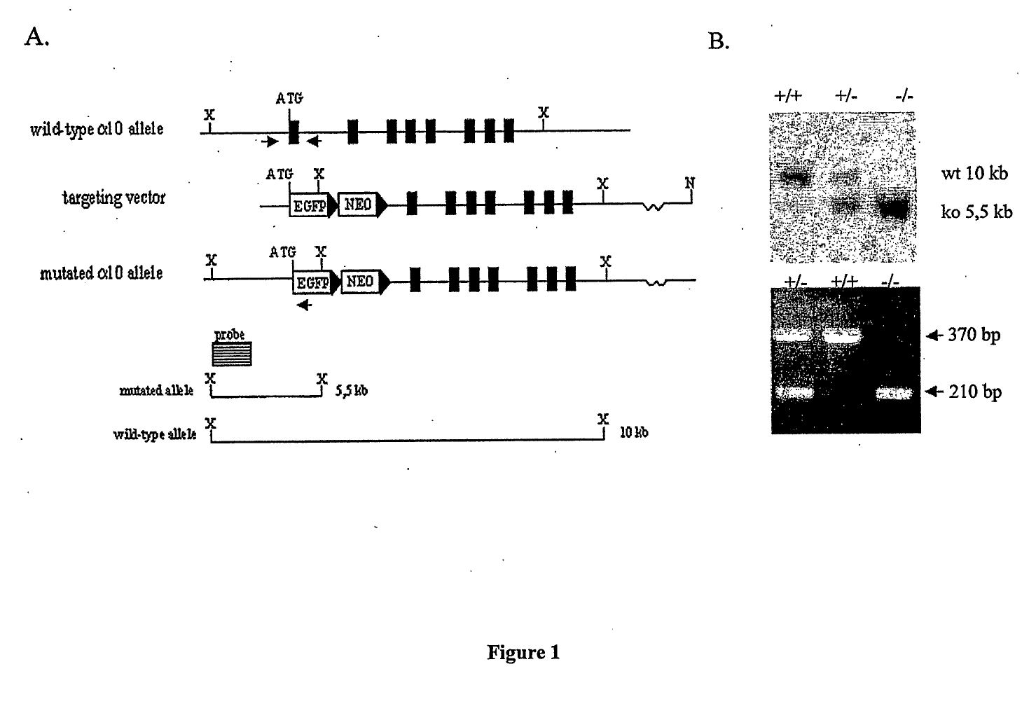

In FIG. 1A, the knockout construct for the alpha10 gene with restriction enzyme map is shown. A vector comprising the nucleic acid sequence encoding the full construct is deposited under the Budapest convention at GSMZ, Germany, under the accession number DSM 14933.

Transfection of the ES Cells and Selection

R1 (Samuel Lunenfeld Research Institute, Room 881, Mount Sinai Hospital, 600 University Avenue, Toronto, Ontario, Canada M5G 1X5) embryonic stem cells were grown on a feeder layer of gamma-irradiated embryonic fibroblast cells in DMEM-media supplemented by 20% heat inactivated calf serum (GIBCO BRL), 0.1 mM beta-mercaptoethanol (Sigma Chemical CO., St Louise, Mo.), non-essential amino acids (GIBCO BRL) and leukemia inhibitory factor (LIF). About 5×10⁷ RI ES cells were electroporated with 60 μg of the Not I-linearized construct, using a Bio-Rad Gene Pulser™ with a setting of 0.8 kV and 3 μf at RT. After 24 hours of growing, selection was started by adding 500 μg/ml G418 (GIBCO BRL) to the medium. After about 7 days, 320 clones were picked and transferred to 24-well plates for additional growth of 3-5 days. Half of each well was then frozen and half was used to isolate DNA for Southern blot analysis.

Southern Blot and PCR for Genotyping

Southern blots were performed from ES cell DNA or tail DNA and digested with the restriction enzyme Xba I. The results are shown in FIG. 1B, top. DNA was run by electrophoresis on a 0.7% agarose gel and probed by a randomly primed ³²P-labelled 0.6 kb probe, generated with the primers 5′-tacagcaggcgaatgaagg (forward) and 5′-aacttaagcttcccctacc (reverse) in a touchdown PCR amplification starting at 66° C. for 12 cycles with −1° C./cycle and then further amplification for 13 cycles at 55° C.

PCR on tail DNA was performed with the primers 5′-acgaaaacagccaaggagagg (forward), 5′-tggtgcagatgaacttcaggg (reverse mutant) and 5′-agagacacgtctcttttctgtg (reverse wild-type) in a touchdown PCR amplification beginning at 68° C. with decreasing 1° C./cycle for 8 cycles and then additionally 60° C. for 35 cycles. This amplification yields a wild-type band of 370 bp and a mutant fragment of 210 bp. The results are shown in FIG. 1B, bottom.

Generation of Alpha 10 Integrin-Deficient Mice and Chimeric Mice

Blastocysts were isolated at day 3.5 post copulation from C57BL/6 mice and two ES clones (about 15 ES cells) were injected (Fassler and Meyer (1995) Genes Dev. 9(15):1896-1908) and transferred to the uterus of 2.5 days post copulation pseudopregnant C57BL/6 females. Chimeric males were mated with C57BL/6 females to test for germ line and then mated with C57BL/6 females to generated outbred lines carrying the alpha10-mutated alleles. The alpha10 deficient mice were then mated with deletor mice, carrying the Cre-recombinase, to generate an alpha10 deficient mouse with no Neo gene.

Conclusion of the Targeting Strategy

Birth of mice homozygous for the mutant alpha10 gene was confirmed by either Southern blot analysis or PCR of tail biopsy DNA, as shown in FIG. 1B. The Southern blot analysis showed that the predicted structure for the homozygous allele (5.5 kb) in the −/−mice was confirmed. PCR detection of the targeted allele in tail biopsy DNA using specific primers detected a 210 bp band in the mutant and a 370 bp band in the wild-type.

Confirmation of the Absence of Alpha 10 in Knockout Mice.

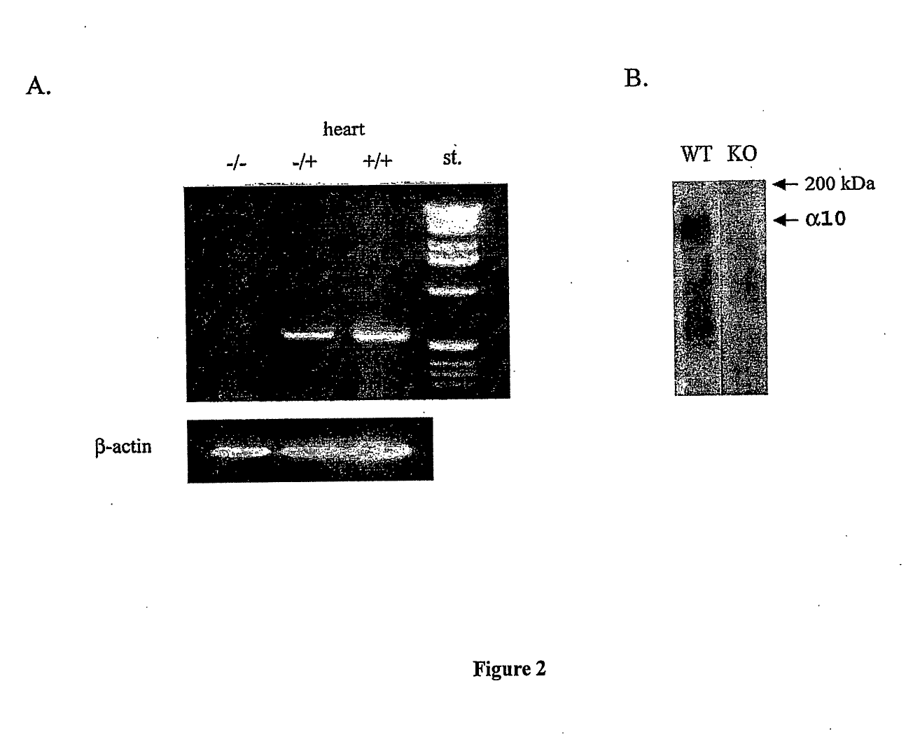

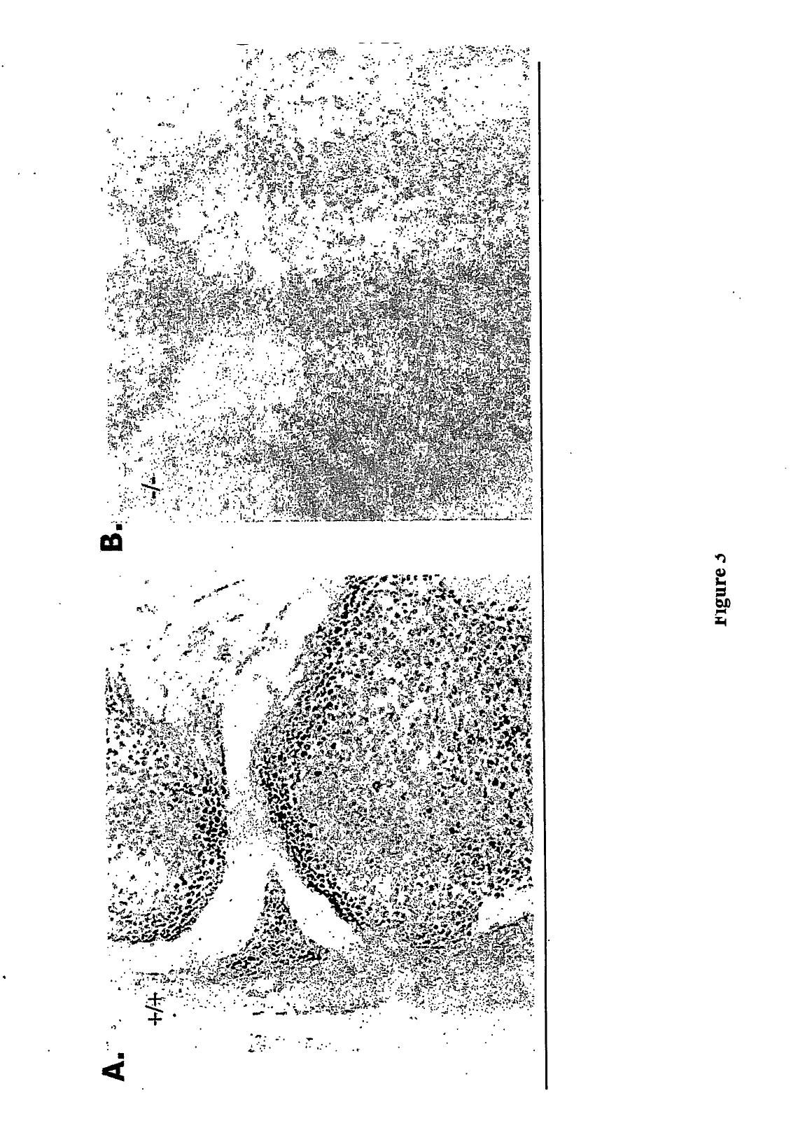

RT-PCR was performed by extracting total RNA from heart by RNeasy Kit (Qiagen, Valencia, Calif., USA) from 4-week old wild-type mice and mice heterozygous and homozygous for the mutated alpha10 gene and the results are shown in FIG. 2A. 3.7 μg of RNA was used to synthesise cDNA, using Superscript™ II Rnase H-Reverse Transcriptase synthesis system (Life Technologies Inc., Grand Island, N.Y., USA).

PCR was run with alpha10 specific primers, 5′-tggagtctctctccatcc (forward, exon 1) and 5′-tcgatgaacagtcttcctaccagc (reverse, exon 6) with a touchdown program starting at 68° C. and decreasing 1° C./cycle for 8 cycles, and then finally amplification at 60° C. for additional 30 cycles. As a positive control, beta-actin specific primers, 5′-gtgggccgctctaggcaccaa (forward) and 5′-ctctttgatgtcacgcacgatttc (reverse) was run in parallel at the same program.

For protein detection of the alpha10 integrin on the cell surface, rib chondrocytes were isolated from newborn wild-type and alpha10 deficient mice as described by Bengtsson et al 2001. The chondrocyte cell surfaces were biotinylated, followed by immunoprecipitation with an antiserum raised against the alpha10 integrin as shown in FIG. 2B, and analysed by Western blotting as described (Bengtsson et al (2001) Matrix Biology 20(8):565-576).

Tissues from wild-type and alpha10 knockout mice were taken at various ages. Tissues for paraffin embedding were fixed in 4% paraformaldehyde (PFA) or Saint-Marie (SM) and limbs were decalcified in 10% EDTA or 10% formic-acid (Sigma-Aldrich) prior to dehydration and embedding. Paraffin sections were then cut at 6 μm on a microtome. Tissue for cryosectioning were snap-frozen, cut at 6 μm and fixed in acetone 5 min at −20° C. prior to staining. All sections containing cartilage were treated with 2 mg/ml hyaluronidase (Sigma) 30 min 37° C. before antibody staining. Immunofluorescent staining and DAB staining (Vectastain ABC-Kit from Vector Laboratories Inc., Burlingham, Calif., USA) was carried out according to standard procedures with the first antibody incubating for 1 hour. The antibody used for recognition of alpha10 was a rabbit polyclonal against the cytoplasmic tail of human alpha10 at a concentration of 1:100 (Camper et al., (1998) J. Biol. Chem. 273(20383-20389).

In FIG. 3, immunohistochemical staining of the knee joint from wild-type (A) and knockout alpha10 mice (B) is shown. Knee joints were stained for the alpha10 subunit by using an immunofluorescent (Cy3) tagged secondary antibody. Alpha10 was detected in the wild-type knee joint (A) but not in the alpha10 knockout knee joint (B).

Conclusion of the Alpha 10 Disruption

The absence of integrin alpha10 mRNA and protein in animals Harboring a homozygous disruption of the integrin gene was confirmed. Total RNA from heart tissue, known to express levels of alpha10, showed no detectable alpha10-specific transcript in the −/− mice. Thus both copies of the alpha10 gene were successfully deleted by the targeting vector.

Results obtained at mRNA level were confirmed at protein level by the absence of immunoreactive alpha10 after surface biotinylation and immunoprecipitation of alpha10 from wild-type and KO mouse chondrocytes.

Further evidence for the absence of alpha10 on chondrocytes was provided by immunohistochemical staining of the knee joints from wild-type and knockout mice. Alpha10 was detected in the +/+ mouse knee joint but not in the −/− knee joint.

Collectively, these results show that disruption of both alleles of the mouse alpha10 gene results in abrogation of the alpha10 mRNA and protein.

Skeletal Staining and Length Measurements of Tibia and Femur

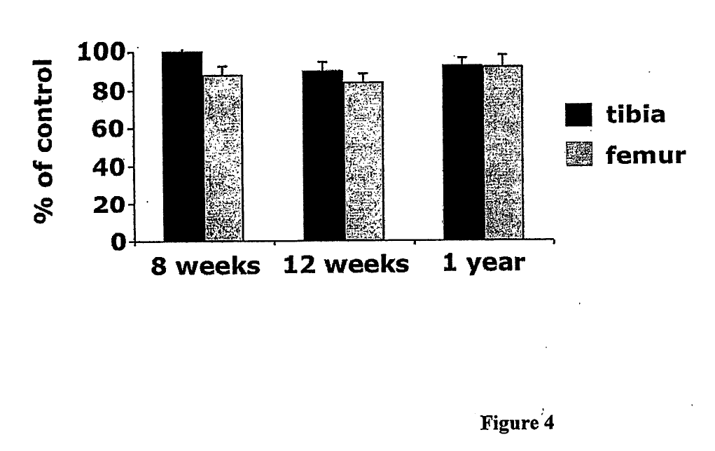

In FIG. 4, length measurements of the tibia and femur is shown in alpha10 knockout mice. Measured length of tibia and femur (at ages 8 weeks, 12 weeks and 1 year) as shown by percent of wild-type length. Alpha10 knockout mice show about 5-10% shorter stature compared to wild-type mice. At 12w, p=0.0007 for femur and p=0.05 for tibia (n=4 for wild-type and n=6 for knockout). At 1 year, p=0.08 for femur and p=0.01 for tibia (n=3 for wild-type and n=4 for knockout).

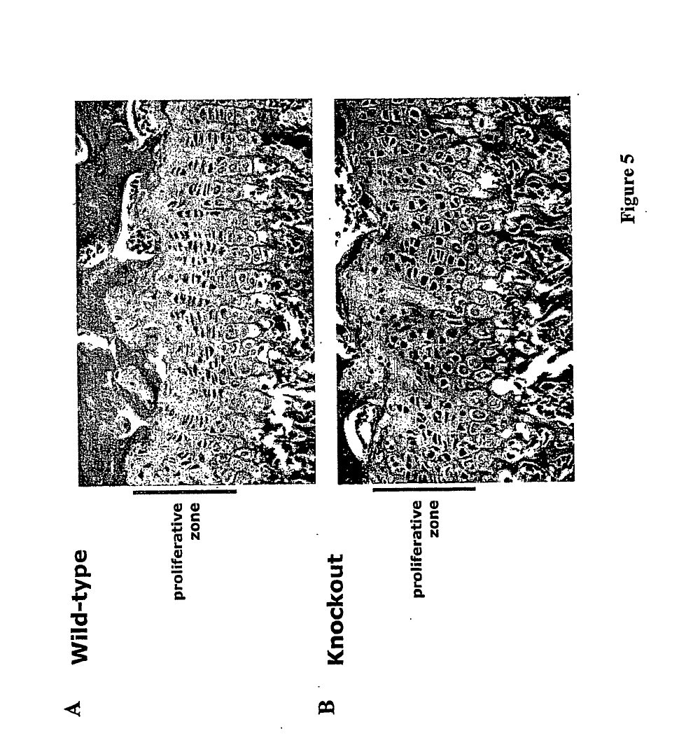

FIG. 5 shows haematoxilin/cytochrome staining of tibial growth plate from wild-type (A) and alpha10 knockout (B) mice. By week 8 the morphology of the chondrocytes in the upper proliferative zone of the growth plate exhibit marked changes in the wild-type (A) compared to the knockout mice (B). Chondrocytes from the knockout mice are irregular in size and are rounder in shape compared to the wild-type. In addition, the regular columnar stacking of the chondrocytes in the wild-type is lost in favour of a more random, disorganised proliferative zone.

Alterations in Bone and Cartilage Morphology

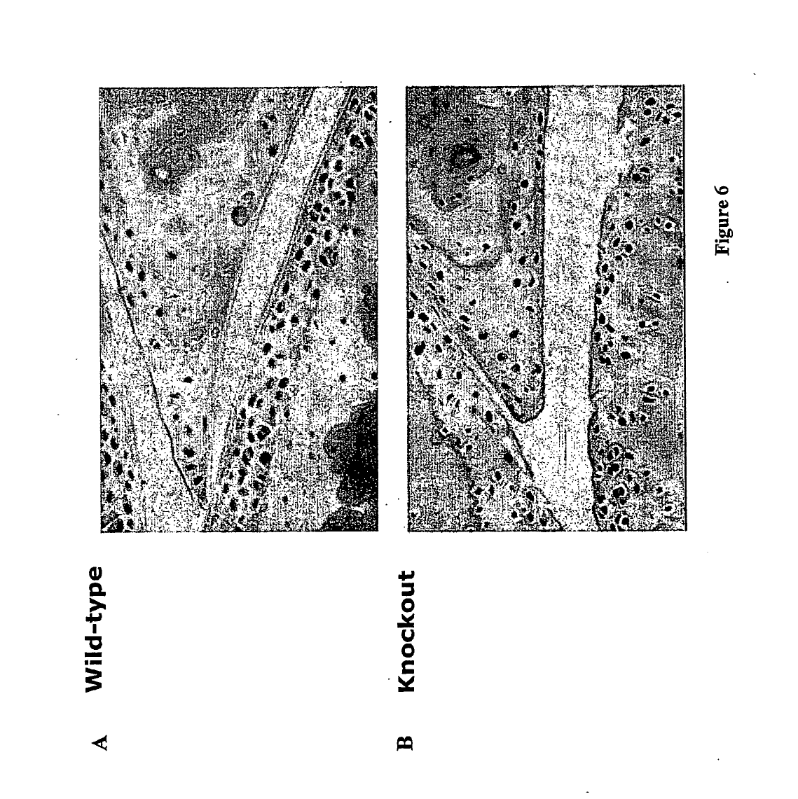

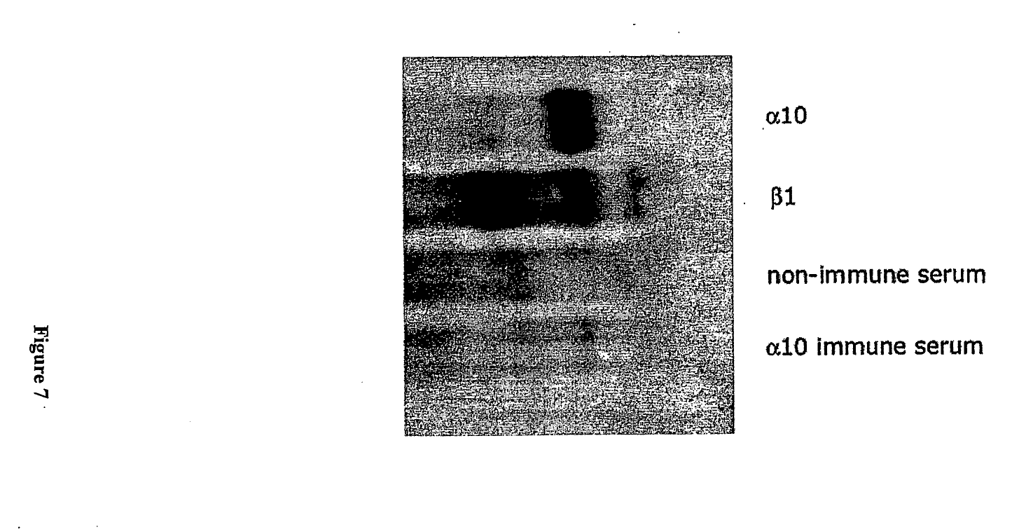

In FIG. 6, haematoxylin/eosin staining of the knee joint from wild-type (A) and alpha10 knockout (B) mice is shown. Gross macroscopic changes resembling early fibrillation are observed in the articular cartilage of 1-year-old knockout mice (B). In contrast, wild-type mice (A) of the same age exhibit a normal, unfibrillated articular surface.

### Example 2

### Disrupting the Integrin Alpha11 Gene

Integrin Alpha 11 Knockout Mouse—Targeting Strategy

Four BAC clones were identified from a spotted 129/SvJ genomic library (BAC Mouse Release II, Genome Systems Inc, St. Louis, Mo.) using a 0.95 kb probe from the 5′end of human alpha11 cDNA (nt-37-917) (Velling, T., Kusche-Gullberg, M., Sejersen, T., and Gullberg, D. (1999) J. Biol. Chem. 274(36), 25735-25742), JBC 274:25735-42. BAC clones were obtained from Genome Systems and after cleavage with Sac I, a 12 kb fragment reactive with the 0.95 kb probe was identified, subcloned into pBluescript SK(+) cloning vector and sequenced (MWG Biotech AG, Germany).

From the 12 kb genomic fragment of mouse itga11, homology arms were excised using NheI to obtain a 5.5 kb long homology arm, and BsaWI+PaeI to obtain a 2.5 kb short homology arm. The resulting gap between homology arms gave a 375 bp deletion beginning with the last 37 nucleotides of exon 3, and extending into intron 3. The targeting construct was made by ligating the short arm to an IRES-lacZ reporter cassette, followed by a lox-p flanked PGKneo cassette ligated in the reverse direction, and ending with the long arm.

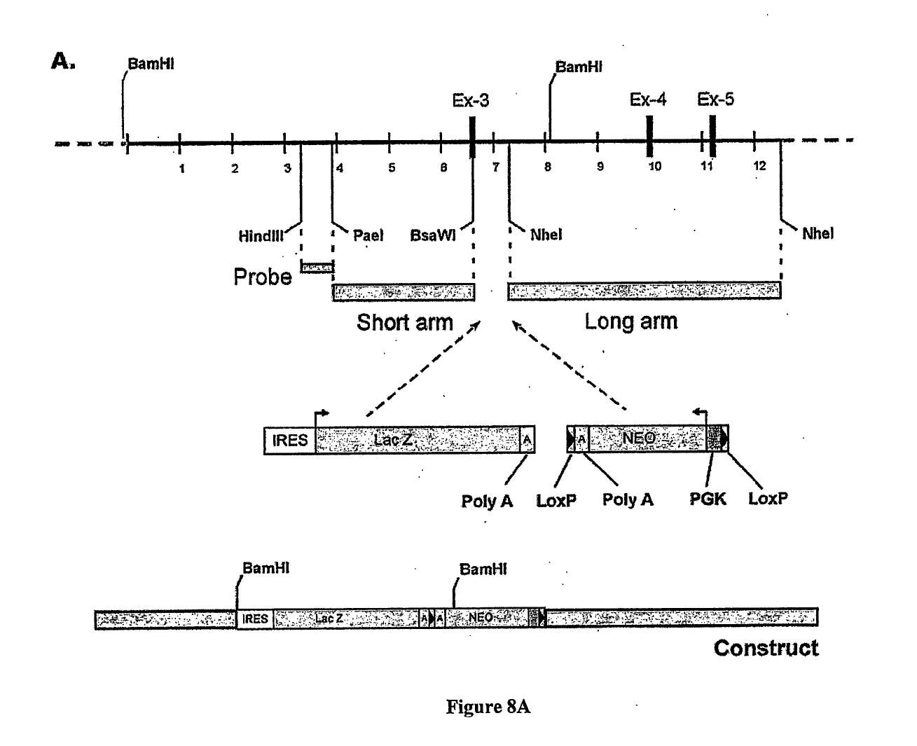

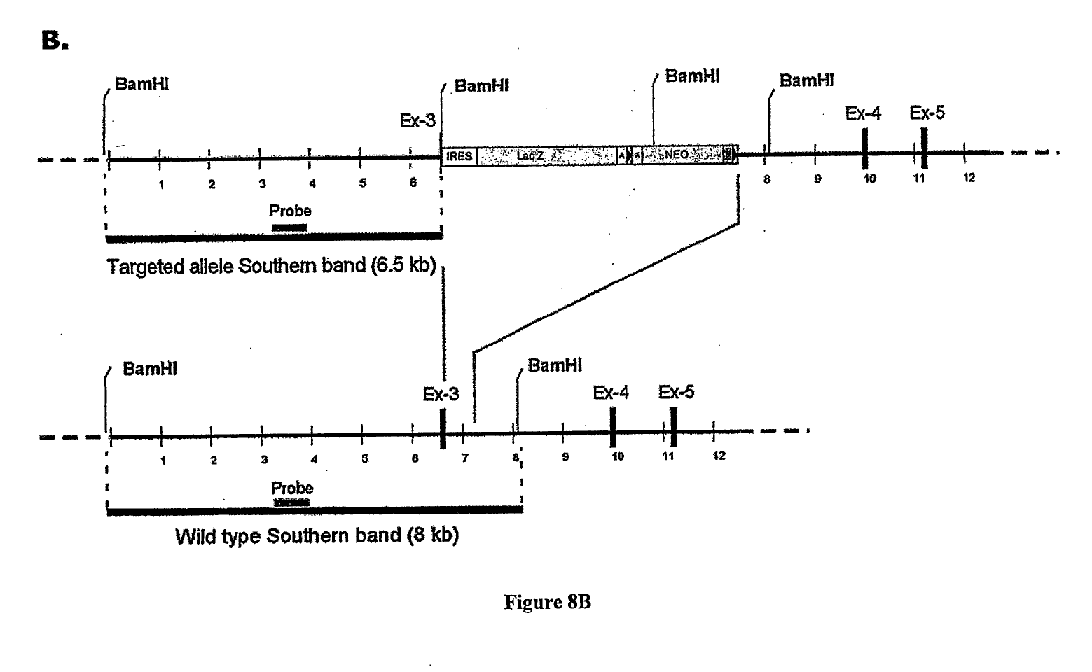

The construct is outlined in FIG. 8A. In FIG. 8B, the restriction enzyme map shows the important sites in the generation of the targeted alpha11 allele.

Southern Blot for Genotyping

Targeted alleles were detected by BamHI digestion of genomic DNA followed by Southern blotting using a 714 bp HindIII/PaeI fragment immediately upstream of the short homology arm as a hybridization probe. The targeted alleles were identified by a shorter band formed (6.5 kb versus 8 kb for the wild-type) due to the introduction of a novel BamHI site from the targeting construct.

Generation of Alpha 11 Integrin-Deficient Mice and Chimeric Mice

Blastocysts were isolated at day 3.5 as above.

The embryonic stem cell line used was R1 (Samuel Lunenfeld Research Institute, Room 881, Mount Sinai Hospital, 600 University Avenue, Toronto, Ontario, Canada M5G 1X5). Cell culture, homologous recombination and microinjection of ES cell clones into C57BL/6J blastocysts were carried out as described in example 1. The targeting construct was linearized with Not I. The mutant heterozygous mice were obtained by crossing chimeric mice to C57BL/6J mice. Heterozygotes (F1) were intercrossed to obtain the homozygous (F2) mice. The genotype of the ES clones was determined by Southern blotting and the genotype of the mice by Southern blot or genomic PCR. In phenotypic analyses, F2 littermates of all three genotypes were used. For analysis of protein expression on fibroblasts and immunohistochemistry on embryos sections, F3 littermates were used.

Confirmation of the Absence of Alpha 11 in Knockout Mice

Tail DNA was prepared as described (Kogan, S. C., Doherty, M., and Gitschier, J. (1987) N Engl J Med 317(16), 985-90) and subjected to PCR in a 20 μl reaction volume with 4 mM MgCl₂, 5% DMSO and 5 pM of each primer, using a mix of 4 primers. Primer sequences were as follows: alpha11 exon 3F: 5′ caactgcaccaagctcaacct 3′; alpha11 intron 3R: 5′ ttctgctgtcacttcctcatc 3′; LacZF 5′ gagcgtggtg gttatgccga tcgc 3′; LacZR 5′ ccga accatccgct gtggta 3′.

DNA was initially denatured for 3 min at 95° C., followed by 30 cycles of 95° C., 1 min; 60° C., 1 min; 72° C., 1 min and a final elongation at 72° C. for 5 min. The reaction was separated on 2% agarose and yielded a 780 bp wild-type band and/or 374 bp KO band.

Conclusion of the Alpha 11 Disruption

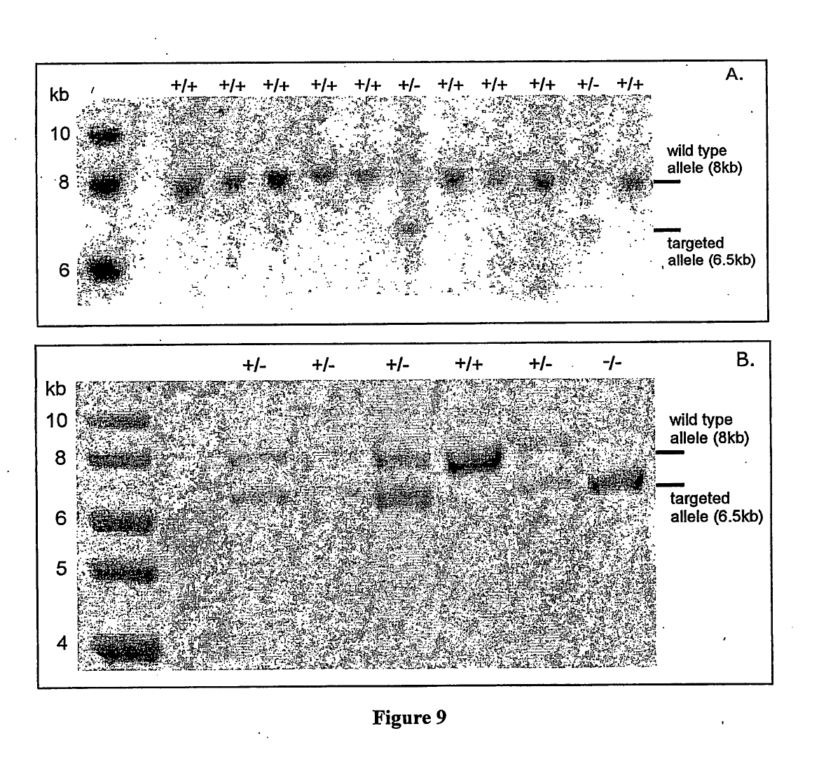

The disruption of the gene locus in mouse ES cells resulted in the generation of an integrin alpha11-deficient mouse. Birth of mice homozygous for the mutant alpha11 gene was confirmed by Southern of DNA isolated from mice tail biopsies. In FIG. 9, Southern blot analysis of DNA isolated from wild-type (+/+), heterozygous (+/−) and homozygous (−/−) alpha11 mice is shown. The wild-type allele and the homozygously targeted alpha11 alleles give rise to an 8 kb and a 6.5 kb fragment in Southern blot, respectively. FIG. 9A shows ES cells on a Southern blot, where the data shows non-recombinant wild-type ES-clones (+/+) and recombinant heterozygous ES-clones (+/−). FIG. 9B shows a Southern blot of mouse tails where the F2-generation shows wild-type (+/+), and mice heterozygous (+/−) and homozygous (−/−) for the targeted allele.

Weight Curves in the Alpha 11 Targeted Mouse

At 3 weeks of age, when the tails were cut for genotyping, it was noted that each litter contained mice that were smaller than their littermates. Mice established from both ES cell clones 95 and 215 were weighed at different ages and the data was assembled into separate growth curves for female and male wt, heterozygous and homozygous littermates. Both female and male homozygous mice revealed on average a 20-30% reduction in size that persisted into adulthood. The overall size of the skeleton was smaller.

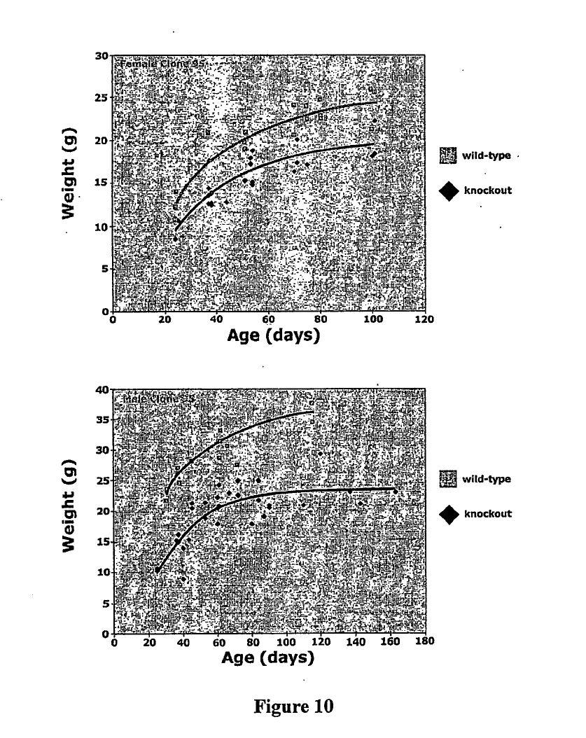

F2-offspring from two separate mouse strains originating from chimera from two different heterozygous embryonic stem-cell clones (clone 95 and 215) were genotyped and body weight was measured at different time points for female and male wild-type and knockout mice. Results are shown for clone 95 only in FIGS. 10A and B. For each sex the number of individuals used is indicated together with the number of measured data points. Female Clone 95 includes wild-type (7 mice, 19 data points) and knockout (11 mice 24 data points). The male Clone 95 includes wild-type (11 mice 30 data points) and knockout (9 mice 28 data points).

Conclusion for Phenotypes

Mice deficient for integrin alpha11 show a significant reduction in body weight (and size, data not shown) corresponding to about 70-80% of the weight of wild-type and heterozygous litter mates.

Changes in Dermal Collagen Content of the Intercapsular Skin in Wild-Type and Knockout Alpha 11 Mice.

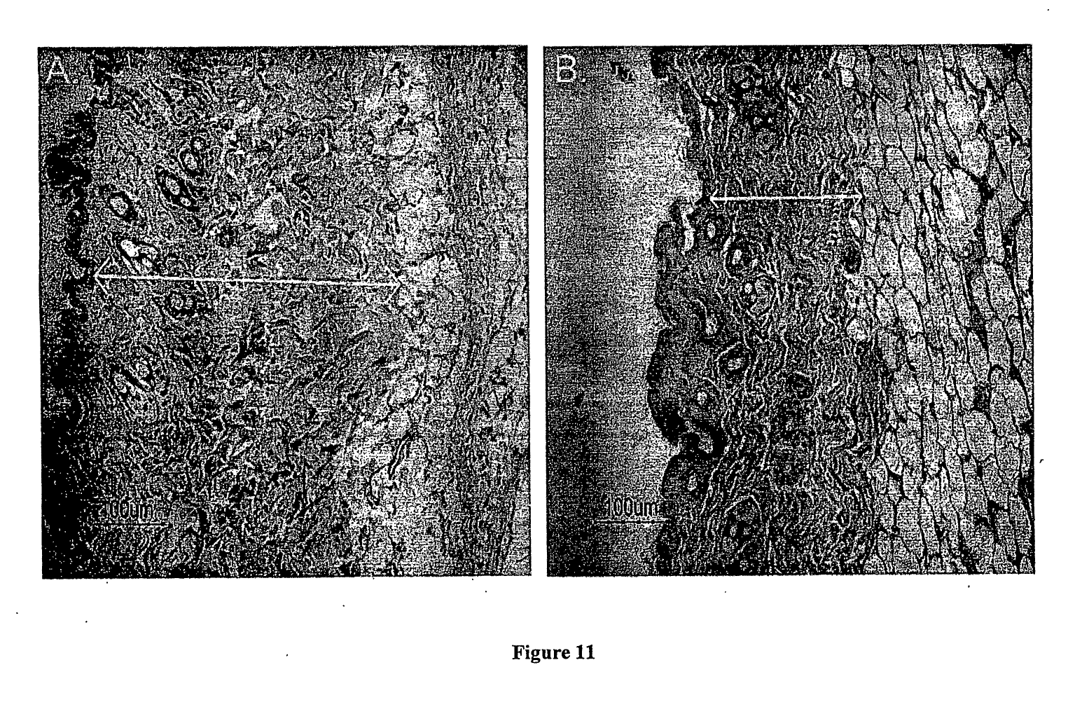

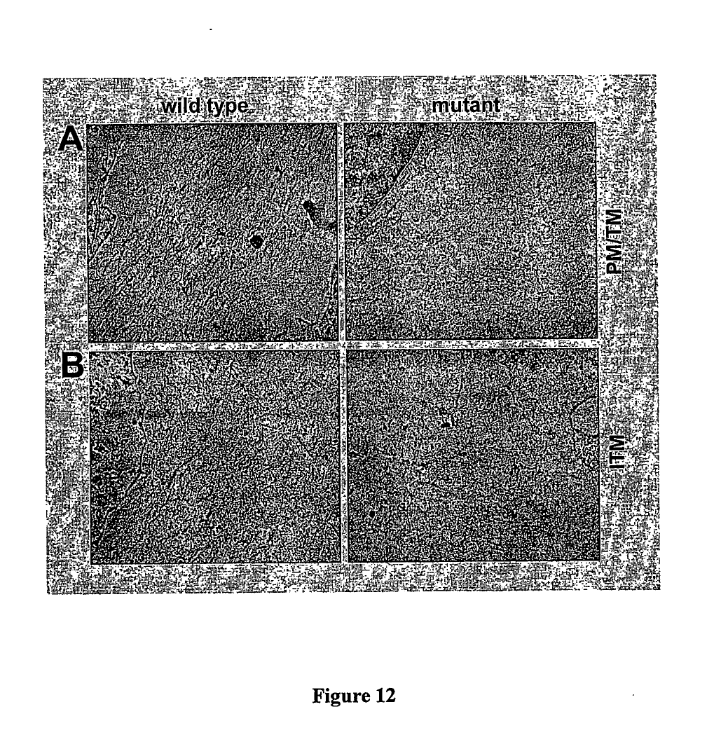

Dermal collagen thickness in intercapsular skin was measured in wild-type (A, FIG. 11) and knockout (B, FIG. 11) mice. As could be seen in FIG. 11, the dermal thickness is reduced in the knockout by about 50% compared to the wild-type.

Depositions

The following depositions have been made according to the Budapest convention in Germany at the Deutsche Sammlung von Mikroorganismen und Zellkulturen GmbH (DSMZ).

A vector comprising the DNA encoding the alpha10 knock-out construct and one vector comprising the DNA encoding the alpha11 knock-out construct respectively, is deposited.

The DSMZ-Accession Number is:

Alpha 10 knockout vector pTN 6.2 comprising the DNA encoding the alpha10 knockout construct: DSM 14933.
Alpha 11 knockout vector 1.1 comprising the DNA encoding the alpha11 knockout construct: DSM 14934.

1. A non-human mammal and its progeny comprising an integrin alpha10 gene, wherein at least a part of an integrin alpha10 gene of said non-human mammal and its progeny has been replaced with a DNA sequence comprising at least a portion of the integrin alpha10 coding sequence linked to a selection marker sequence.

2. The non-human mammal and its progeny according to claim 1, wherein the portion of the integrin alpha10 coding sequence is at least a portion of the integrin alpha10 intron, exon, promotor or a mixture thereof.

3. The non-human mammal and its progeny according to claim 1, wherein the DNA sequence comprising at least a portion of one exon of the integrin alpha10 coding sequence linked to a selection marker sequence deposited with the Deutsche Sammlung von Mikroorganismen und Zellkulturen GmbH, Germany, under the accession number DSM 14933.

4. A non-human mammal and its progeny comprising an integrin alpha11 gene, wherein at least a part of an integrin alpha11 gene of said non-human mammal and its progeny has been replaced with a DNA sequence comprising at least a portion of the integrin alpha11 coding sequence linked to a selection marker sequence.

5. The non-human mammal and its progeny according to claim 4, wherein the portion of the integrin alpha11 coding sequence is at least a portion of the integrin alpha11 intron, exon, promotor or a mixture thereof.

6. The non-human mammal and its progeny according to claim 4, wherein the DNA sequence comprising at least a portion of one exon of the integrin alpha11 coding sequence linked to a selection marker sequence deposited with the Deutsche Sammlung von Mikroorganismen und Zellkulturen GmbH, Germany, under the accession number DSM 14934.

7. A non-human mammal and its progeny comprising an integrin alpha10 gene and an integrin alpha11 gene, wherein at least a part of an integrin alpha10 gene of said non-human mammal and its progeny has been replaced with 1) a DNA sequence comprising at least a portion of the integrin alpha10 coding sequence linked to a first marker sequence and 2) a DNA sequence comprising at least a portion of the integrin alpha11 coding sequence linked to a second selection marker sequence.

8. A knockout non-human mammal and its progeny, wherein expression of the gene encoding integrin alpha10 is suppressed as compared to a wild type non-human mammal.

9. The knockout non-human mammal and its progeny according to claim 8, wherein the suppression of the gene encoding integrin alpha10 is 100%.

10. The knockout non-human mammal and its progeny according to claim 8, wherein the alpha10 is suppressed through insertion of a knockout construct comprising at least a portion of the integrin alpha10 coding sequence linked to a selection marker sequence.

11. The non-human mammal and its progeny according to claim 10, wherein the portion of the integrin alpha10 coding sequence is at least a portion of the integrin alpha10 intron, exon, promotor or a mixture thereof.

12. The non-human mammal and its progeny according to claim 10, wherein the knockout construct comprises at least a portion of one exon of the integrin alpha10 coding sequence linked to a selection marker sequence, and is deposited with the Deutsche Sammlung von Mikroorganismen und Zellkulturen GmbH, Germany, under the accession number DSM 14933.

13. A knockout non-human mammal and its progeny, wherein expression of the gene encoding integrin alpha11 is suppressed as compared to a wild-type non-human mammal.

14. The knockout non-human mammal and its progeny according to claim 13, wherein the suppression of the gene encoding integrin alpha11 is 100%.

15. The knockout non-human mammal and its progeny according to claim 13, wherein the alpha11 is suppressed through insertion of a knockout construct comprising at least a portion of the integrin alpha11 coding sequence linked to a marker sequence.

16. The non-human mammal and its progeny according to claim 15, wherein the portion of the integrin alpha11 coding sequence is at least a portion of the integrin alpha11 intron, exon, promotor or a mixture thereof.

17. The non-human mammal and its progeny according to claim 16, wherein the knockout construct comprises at least a portion of one exon of the integrin alpha11 coding sequence linked to a marker sequence, and is deposited with the Deutsche Sammlung von Mikroorganismen und Zellkulturen GmbH, Germany, under the accession number DSM 14934.

18. A knockout non-human mammal and its progeny, wherein expression of the gene encoding integrin alpha10 and the gene encoding integrin alpha11 is suppressed as compared to a wild type non-human mammal.

19. The knockout non-human mammal and its progeny according to claim 18, wherein the suppression of the genes encoding integrin alpha10 and alpha11 is 100%.

20. The knockout non-human mammal and its progeny according to claim 18, wherein the alpha10 is suppressed through insertion of a knockout construct comprising at least a portion of the integrin alpha10 coding sequence linked to a selection marker sequence, and wherein the alpha11 is suppressed through insertion of a knockout construct comprising at least a portion of the integrin alpha11 coding sequence linked to a second selection marker sequence.

21. The non-human mammal and its progeny according to claim 20, wherein the portion of the integrin alpha10 coding sequence is at least a portion of the integrin alpha10 intron, exon, promotor or a mixture thereof, and wherein the portion of the integrin alpha11 coding sequence is at least a portion of the integrin alpha11 intron, exon, promotor or a mixture thereof.

22. The non-human mammal and its progeny according to claim 20, wherein the knockout construct comprises at least a portion of one exon of the integrin alpha10 coding sequence linked to a marker sequence, and is deposited with the Deutsche Sammlung von Mikroorganismen und Zellkulturen GmbH, Germany, under the accession number DSM 14933, and wherein the knockout construct comprises at least a portion of one exon of the integrin alpha11 coding sequence linked to a marker sequence, and is deposited with the Deutsche Sammlung von Mikroorganismen und Zellkulturen GmbH, Germany, under the accession number DSM 14934.

23. The non-human mammal and its progeny according to claim 1, wherein the non-human mammal and its progeny is a rodent.

24. The non-human mammal and its progeny according to claim 23, wherein the rodent is a mouse.

25. The non-human mammal and its progeny according to claim 1, wherein the selection marker sequence is the neomycin resistance gene.

26. A method for preparing a non-human mammal and its progeny with a disrupted integrin alpha10 gene, comprising the step of replacing a portion of the integrin alpha10 gene in an embryonic stem cell by homologous recombination with a DNA sequence comprising at least a portion of one exon of the integrin alpha10 coding sequence linked to a marker sequence.

27. The method according to claim 26, further comprising the steps of
a) providing a knockout construct for integrin alpha10,
b) providing an ES cell line,
c) transforming the ES cell line in b) with the construct in a),
d) selecting transformed ES cell line using a marker sequence,
e) providing a blastocyst,
f) introducing the ES cell line into the blastocyst,
g) transferring the blastocyst to a fostermother non-human mammal, and,
h) allowing an embryo to develop to a chimaeric animal to enable germline transmission of the disrupted integrin alpha10 gene.

28. A vector comprising nucleic acid sequence encoding the integrin alpha10 knockout construct, wherein the sequence comprises at least a portion of one exon of the integrin alpha10 coding sequence linked to a selection marker sequence, deposited with the Deutsche Sammlung von Mikroorganismen und Zellkulturen GmbH, Germany, under the accession number DSM 14933.

29. An ES cell line comprising the integrin alpha10 knockout construct vector according to claim 28.

30. A method for preparing a non-human mammal and its progeny with a disrupted integrin alpha11 gene, comprising the step of replacing a portion of the integrin alpha11 gene in an embryonic stem cell by homologous recombination with a DNA sequence comprising at least a portion of one exon of the integrin alpha11 coding sequence linked to a selection marker sequence.

31. The method according to claim 30, further comprising the steps of
i) providing a knockout construct for integrin alpha11,
j) providing an ES cell line,
k) transforming the ES cell line in j) with the construct in i),
l) selecting transformed ES cell line using a marker sequence,
m) providing a blastocyst,
n) introducing the ES cell line into the blastocyst,
o) transferring the blastocyst to a fostermother non-human mammal, and,
p) allowing an embryo to develop to a chimaeric animal to enable germline transmission of the disrupted integrin alpha11 gene.

32. A vector comprising nucleic acid sequence encoding the integrin alpha11 knockout construct, wherein the sequence comprises at least a portion of one exon of the integrin alpha11 coding sequence linked to a selection marker sequence, deposited with the Deutsche Sammlung von Mikroorganismen und Zellkulturen GmbH, Germany, under the accession number DSM 14934.

33. An ES cell line comprising the integrin alpha11 knockout construct vector according to claim 32.

34. A method for preparing a non-human mammal and its progeny with a disrupted integrin alpha10 gene and a disrupted integrin alpha11 gene, comprising the steps of
q) replacing a portion of the integrin alpha10 gene in an embryonic stem cell by homologous recombination with a DNA sequence comprising at least a portion of one exon of the integrin alpha10 coding sequence linked to a selection marker sequence, and,
r) replacing a portion of the integrin alpha11 gene in an embryonic stem cell by homologous recombination with a DNA sequence comprising at least a portion of one exon of the integrin alpha11 coding sequence linked to a selection marker sequence.

35. The method according to claim 34, further comprising the steps of
s) providing a knockout construct for integrin alpha10, and providing a knockout construct for integrin alpha11,
t) providing an ES cell line,
u) transforming the ES cell line in t) with the constructs in s), either at the same time or one at a time,
v) selecting transformed ES cell line using at least one marker sequence,
w) providing a blastocyst,
x) introducing the transformed and selected ES cell line into the blastocyst,
y) transferring the blastocyst to a fostermother non-human mammal, and,
z) allowing an embryo to develop to a chimaeric animal to enable germline transmission of the disrupted integrin alpha10 gene.

36. A method for modulating activity in musculoskeletal and connective tissue diseases comprising utilizing the non-human mammal and its progeny according to claim 1 as a model.

37. A method for modulating activity in atherosclerosis comprising utilizing the non-human mammal and its progeny according to claim 1 as a model.

38. A method for modulating activity in fibrosis comprising utilizing the non-human mammal and its progeny according to claim 1 as a model.

39. A method for modulating activity in differentiation or function of a stem cell comprising utilizing the non-human mammal and its progeny according to claim 1 as a model.

40. The method according to claim 39, wherein the stem cell is selected from the group consisting of a mesenchymal stem cell, haematopoetic stem cell, and epithelial stem cell.

41. A method for modulating activity in bone fracture healing comprising utilizing the non-human mammal and its progeny according to claim 1 as a model.

42. A method for modulating activity in inflammatory diseases comprising utilizing the non-human mammal and its progeny according to claim 1 as a model.

43. The method according to claim 42, wherein the inflammatory disease is rheumatoid arthritis or meningitis.

44. A method for modulating activity of alpha10 and/or alpha11 comprising utilizing the non-human mammal and its progeny according to claim 1 as a gene therapeutic model.

45. A method for modulating activity in heart valve diseases comprising utilizing the non-human mammal and its progeny according to claim 1 as a model.

46. The method according to claim 36, wherein modulating activity is preventing, inhibiting, alleviating or reversing activity in said diseases.

47. The method according to claim 41, wherein modulating activity is stimulating, preventing, inhibiting, alleviating or reversing activity in bone fracture healing.

48. A method for the generation of antibodies showing reactivity with the integrin alpha10-beta1, alpha11-beta1 or parts thereof comprising utilizing the non-human mammal and its progeny according to claim 25.

49. The method according to claim 48, wherein the antibodies are polyclonal antibodies.

50. The method according to claim 48, wherein the antibodies are monoclonal antibodies.

51. The method according to claim 48, wherein the integrin alpha10-beta1, alpha11-beta1 or parts thereof is human integin alpha10-beta1, alpha11-beta1 or parts thereof.

52. A method for screening agents for effects in musculoscleletal diseases, comprising the steps of
i) providing a knockout mouse according to claim 1,
ii) administering a test agent to said knockout mouse,
iii) determining the effect of said test agent on morphology, histology, synthesis and degradation of extracellular matrix molecules, and synthesis of integrins, and
iv) correlating the effect of said test agent in iii) above with musculoskeletal and connective tissue diseases.

53. A method for screening agents for effects in atherosclerosis, comprising the steps of
i) providing a knockout mouse according claim 1,
ii) administering a test agent to said knockout mouse,
iii) determining the effect of said test agent on morphology, histology, synthesis and degradation of extracellular matrix molecules, and synthesis of integrins, and
iv) correlating the effect of said test agent in iii) above with atherosclerosis.

54. A method for screening agents for effects in fibrosis, comprising the steps of
i) providing a knockout mouse according to claim 1,
ii) administering a test agent to said knockout mouse,
iii) determining the effect of said test agent on morphology, histology, synthesis and degradation of extracellular matrix molecules, and synthesis of integrins, and
iv) correlating the effect of said test agent in iii) above with fibrosis.

55. A method for screening agents for effects in differentiation of stem cells, comprising the steps of
i) providing a knockout mouse according to claim 1,
ii) administering a test agent to said knockout mouse,
iii) determining the effect of said test agent on morphology, histology, synthesis and degradation of extracellular matrix molecules, and synthesis of integrins, and
iv) correlating the effect of said test agent in iii) above with differentiation of stem cells.

56. A method for screening agents for effects in fracture healing, comprising the steps of
i) providing a knockout mouse according to claim 1,
ii) administering a test agent to said knockout mouse,
iii) determining the effect of said test agent on morphology, histology, synthesis and degradation of extracellular matrix molecules, and synthesis of integrins, and
iv) correlating the effect of said test agent in iii) above with fracture healing.

57. A method for screening agents for effects in inflammatory diseases, comprising the steps of
i) providing a knockout mouse according to claim 1,
ii) administering a test agent to said knockout mouse,
iii) determining the effect of said test agent on morphology, histology, synthesis and degradation of extracellular matrix molecules, and synthesis of integrins, and
iv) correlating the effect of said test agent in iii) above with inflammatory diseases.

58. A method for screening agents for effects in gene therapy of alpha 10 and/or alpha 11, comprising the steps of
i) providing a knockout mouse according to claim 1,
ii) administering a test agent to said knockout mouse,
iii) determining the effect of said test agent on morphology, histology, synthesis and degradation of extracellular matrix molecules, and synthesis of integrins, and
iv) correlating the effect of said test agent in iii) above with expression of alpha 10 and/or alpha 11.

59. The method according to claim 58, wherein the test agent is selected from the group consisting of deoxynucleic acid, ribonucleic acid, PNA, and mixtures thereof.

60. A method for generating antibody-producing hybridomas reactive to integrin alpha10-beta1 or alpha11-beta1 or parts thereof, comprising the steps of
i) immunizing a mouse with integrin alpha10-beta1 or parts thereof, or integrin alpha10-beta1 or parts thereof,
ii) boosting the immunized mouse in i) above with the immunized antigen,
iii) sacrifying the immunized mouse,
iv) preparing single cell suspension cells from the sacrificed mouse in iii),
vii) fusing the single cell suspension in iv) above with a tumour cell line to generate hybridoma cells,
viii) screening the hybridoma cells for reactivity to the immunized antigen.

61. The method in claim 60, further comprising the steps of limiting dilution of screened, fused hybridoma cells in vi) above, thereby generating monoclonal hybridoma cells.


SEQUENCE LISTING (18 sequences)

SEQ ID NO: 1 - protein, 5 aa, Artificial Sequence
  Description of Artificial Sequence Synthetic peptide motif
GFFKR

SEQ ID NO: 2 - protein, 7 aa, Artificial Sequence
  Description of Artificial Sequence Synthetic peptide motif
KXGFFXR

SEQ ID NO: 3 - protein, 7 aa, Artificial Sequence
  Description of Artificial Sequence Synthetic peptide
KLGFFAH

SEQ ID NO: 4 - protein, 5 aa, Artificial Sequence
  Description of Artificial Sequence Synthetic peptide
FGGAP

SEQ ID NO: 5 - protein, 5 aa, Artificial Sequence
  Description of Artificial Sequence Synthetic peptide
GFFRS

SEQ ID NO: 6 - DNA, 19 bp, Artificial Sequence
  Description of Artificial Sequence Synthetic primer
tacagcaggcgaatgaagg

SEQ ID NO: 7 - DNA, 19 bp, Artificial Sequence
  Description of Artificial Sequence Synthetic primer
aacttaagcttcccctacc

SEQ ID NO: 8 - DNA, 21 bp, Artificial Sequence
  Description of Artificial Sequence Synthetic primer
acgaaaacagccaaggagagg

SEQ ID NO: 9 - DNA, 21 bp, Artificial Sequence
  Description of Artificial Sequence Synthetic primer
tggtgcagatgaacttcaggg

SEQ ID NO: 10 - DNA, 22 bp, Artificial Sequence
  Description of Artificial Sequence Synthetic primer
agagacacgtctcttttctgtg

SEQ ID NO: 11 - DNA, 18 bp, Artificial Sequence
  Description of Artificial Sequence Synthetic primer
tggagtctctctccatcc

SEQ ID NO: 12 - DNA, 24 bp, Artificial Sequence
  Description of Artificial Sequence Synthetic primer
tcgatgaacagtcttcctaccagc

SEQ ID NO: 13 - DNA, 21 bp, Artificial Sequence
  Description of Artificial Sequence Synthetic primer
gtgggccgctctaggcaccaa

SEQ ID NO: 14 - DNA, 24 bp, Artificial Sequence
  Description of Artificial Sequence Synthetic primer
ctctttgatgtcacgcacgatttc

SEQ ID NO: 15 - DNA, 21 bp, Artificial Sequence
  Description of Artificial Sequence Synthetic primer
caactgcaccaagctcaacct

SEQ ID NO: 16 - DNA, 21 bp, Artificial Sequence
  Description of Artificial Sequence Synthetic primer
ttctgctgtcacttcctcatc

SEQ ID NO: 17 - DNA, 24 bp, Artificial Sequence
  Description of Artificial Sequence Synthetic primer
gagcgtggtggttatgccgatcgc

SEQ ID NO: 18 - DNA, 20 bp, Artificial Sequence
  Description of Artificial Sequence Synthetic primer
ccgaaccatccgctgtggta